(19) United States
(12) Patent Application Publication
(10) Pub. No.: US 2003/0092629 A1
(43) Pub. Date: May 15, 2003

(54) Inhibitors of memapsin 2 and use thereof

(75) Inventors: Jordan J. N. Tang, Edmond, OK (US); Gerald Koelsch, Oklahoma City, OK (US); Arun K. Ghosh, River Forest, IL (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Oklahoma Medical Research Foundation, Oklahoma City, OK

(21) Appl. No.: 10/032,818

(22) Filed: Dec. 28, 2001

Related U.S. Application Data
(60) Provisional application No. 60/275,756, filed on Mar. 14, 2001.
(60) Provisional application No. 60/258,705, filed on Dec. 28, 2000.

(51) Int. Cl. A61K 38/10; C07K 7/08

(52) U.S. Cl. 514/13; 530/326

(57) ABSTRACT

Methods for the production of purified, catalytically active, recombinant memapsin 2 have been developed-, The substrate and subsite specificity of the catalytically active enzyme have been determined by a method which determines the initial hydrolysis rate of the substrates by using MALDI-TOF/MS. Alternatively, the subsite specificity of memapsin can be determined by probing a library of inhibitors with memapsin 2 and subsequently detecting the bound memapsin 2 with an antibody raised to memapsin 2 and an alkaline phosphatase conjugated secondary antibody. The substrate and subsite specificity information was used to design substrate analogs of the natural memapsin 2 substrate that can inhibit the function of memapsin 2. The substrate analogs are based on peptide sequences, shown to be related to the natural peptide substrates for memapsin 2. The substrate analogs contain at least one analog of an amide bond which is not capable of being cleaved by memapsin 2. Processes for the synthesis of substrate analogues including isosteres at the sites of the critical amino acid residues were developed and the more than seventy substrate analogues were synthesized, among which MMI-005, MMI-012, MMI-017, MMI-018, MMI-025, MMI-026, MMI-037, MMI-039, MMI-040, MMI-066, MMI-070, and MMI-071 have inhibition constants in the range of 1.4-61.4×10⁻⁹ M against recombinant pro-memapsin 2. These inhibitors are useful in diagnostics and for the treatment and/or prevention of Alzheimer's disease.

### RELATED APPLICATIONS

This application claims the benefit of U.S. Provisional Application No. 60/275,756, filed on Mar. 14, 2001 and 60/258,705 filed on Dec. 28, 2000, the entire teachings of both of which are incorporated by reference in their entirety.

### BACKGROUND OF THE INVENTION

This invention is in the area of the design and synthesis of specific inhibitors of the aspartic protease Memapsin 2 (beta-secretase, or β-secretase) which are useful in the treatment and/or prevention of Alzheimer's Disease.

Alzheimer's disease (AD) is a degenerative disorder of the brain first described by Alios Alzheimer in 1907 after examining one of his patients who suffered drastic reduction in cognitive abilities and had generalized dementia (The early story of Alzheimer's Disease, edited by Bick et al. (Raven Press, New York 1987)). It is the leading cause of dementia in elderly persons. AD patients have increased problems with memory loss and intellectual functions which progress to the point where they cannot function as normal individuals. With the loss of intellectual skills the patients exhibit personality changes, socially inappropriate actions and schizophrenia (A Guide to the Understanding of Alzheimer's Disease and Related Disorders, edited by Jorm (New York University Press, New York 1987)). AD is devastating for both victims and their families, for there is no effective palliative or preventive treatment for the inevitable neurodegeneration.

AD is associated with neuritic plaques measuring up to 200 μm in diameter in the cortex, hippocampus, subiculum, hippocampal gyrus, and amygdala. One of the principal constituents of neuritic plaques is amyloid, which is stained by Congo Red (Fisher (1983); Kelly Microbiol. Sci. 1(9):214-219 (1984)). Amyloid plaques stained by Congo Red are extracellular, pink or rust-colored in bright field, and birefringent in polarized light. The plaques are composed of polypeptide fibrils and are often present around blood vessels, reducing blood supply to various neurons in the brain.

Various factors such as genetic predisposition, infectious agents, toxins, metals, and head trauma have all been suggested as possible mechanisms of AD neuropathy. Available evidence strongly indicates that there are distinct types of genetic predispositions for AD. First, molecular analysis has provided evidence for mutations in the amyloid precursor protein (APP) gene in certain AD-stricken families (Goate et al. Nature 349:704-706 (1991); Murrell et al. Science 254:97-99 (1991); Chartier-Harlin et al. Nature 353:844-846 (1991); Mullan et al., Nature Genet. 1:345-347 (1992)). Additional genes for dominant forms of early onset AD reside on chromosome 14 and chromosome 1 (Rogaev et al., Nature 376:775-778 (1995); Levy-Lahad et al., Science 269:973-977 (1995); Sherrington et al., Nature 375:754-760 (1995)). Another loci associated with AD resides on chromosome 19 and encodes a variant form of apolipoprotein E (Corder, Science 261:921-923 (1993)).

Amyloid plaques are abundantly present in AD patients and in Down's Syndrome individuals surviving to the age of 40. The overexpression of APP in Down's Syndrome is recognized as a possible cause of the development of AD in Down's patients over thirty years of age (Rumble et al., New England J. Med. 320:1446-1452 (1989); Mann et al., Neurobiol. Aging 10:397-399 (1989)). The plaques are also present in the normal aging brain, although at a lower number. These plaques are made up primarily of the amyloid β peptide (Aβ; sometimes also referred to in the literature as β-amyloid peptide or β peptide) (Glenner and Wong, Biochem. Biophys. Res. Comm. 120:885-890 (1984)), which is also the primary protein constituent in cerebrovascular amyloid deposits. The amyloid is a filamentous material that is arranged in beta-pleated sheets. Aβ is a hydrophobic peptide comprising up to 43 amino acids.

The determination of its amino acid sequence led to the cloning of the APP cDNA (Kang et al., Nature 325:733-735 (1987); Goldgaber et al., Science 235:877-880 (1987); Robakis et al., Proc. Natl. Acad. Sci. 84:4190-4194 (1987); Tanzi et al., Nature 331:528-530 (1988)) and genomic APP DNA (Lemaire et al., Nucl. Acids Res. 17:517-522 (1989); Yoshikai et al., Gene 87, 257-263 (1990)). A number of forms of APP cDNA have been identified, including the three most abundant forms, APP695, APP75 1, and APP770. These forms arise from a single precursor RNA by alternate splicing. The gene spans more than 175 kb with 18 exons (Yoshikai et al. (1990)). APP contains an extracellular domain, a transmembrane region and a cytoplasmic domain. Aβ consists of up to 28 amino acids just outside the hydrophobic transmembrane domain and up to 15 residues of this transmembrane domain. Aβ is normally found in brain and other tissues such as heart, kidney and spleen. However, Aβ deposits are usually found in abundance only in the brain.

Van Broeckhaven et al., Science 248:1120-1122 (1990), have demonstrated that the APP gene is tightly linked to hereditary cerebral hemorrhage with amyloidosis (HCHWA-D) in two Dutch families. This was confirmed by the finding of a point mutation in the APP coding region in two Dutch patients (Levy et al., Science 248:1124-1128 (1990)). The mutation substituted a glutamine for glutamic acid at position 22 of the Aβ (position 618 of APP695, or position 693 of APP770). In addition, certain families are genetically predisposed to Alzheimer's disease, a condition referred to as familial Alzheimer's disease (FAD), through mutations resulting in an amino acid replacement at position 717 of the full length protein (Goate et al. (1991); Murrell et al. (1991); Chartier-Harlin et al. (1991)). These mutations co-segregate with the disease within the families and are absent in families with late-onset AD. This mutation at amino acid 717 increases the production of the Aβ₁₋₄₂ form of Aβ from APP (Suzuki et al., Science 264:1336-1340 (1994)). Another mutant form contains a change in amino acids at positions 670 and 671 of the full length protein (Mullan et al. (1992)). This mutation to amino acids 670 and 671 increases the production of total Aβ from APP (Citron et al., Nature 360:622-674 (1992)).

APP is processed in vivo at three sites. The evidence suggests that cleavage at the β-secretase site by a membrane associated metalloprotease is a physiological event. This site is located in APP 12 residues away from the lumenal surface of the plasma membrane. Cleavage of the β-secretase site (28 residues from the plasma membrane's lumenal surface) and the β-secretase site (in the transmembrane region) results in the 40/42-residue β-amyloid peptide (A β), whose elevated production and accumulation in the brain are the central events in the pathogenesis of Alzheimer's disease (for review, see Selkoe, D. J. Nature 399:23-31 (1999)). Presenilin 1, another membrane protein found in human brain, controls the hydrolysis at the APP γ β-secretase site and has been postulated to be itself the responsible protease (Wolfe, M. S. et al., Nature 398:513-517 (1999)). Presenilin 1 is expressed as a single chain molecule and its processing by a protease, presenilinase, is required to prevent it from rapid degradation (Thinakaran, G. et al., Neuron 17:181-190 (1996) and Podlisny, M. B., et al., Neurobiol. Dis. 3:325-37 (1997)). The identity of presenilinase is unknown. The in vivo processing of the β-secretase site is thought to be the rate-limiting step in Aβ production (Sinha, S. & Lieberburg, I., Proc. Natl. Acad. Sci., USA, 96:11049-11053 (1999)), and is therefore a strong therapeutic target.

The design of inhibitors effective in decreasing amyloid plaque formation is dependent on the identification of the critical enzyme(s) in the cleavage of APP to yield the 42 amino acid peptide, the Aβ₁₋₄₂ form of Aβ. Although several enzymes have been identified, it has not been possible to produce active enzyme. Without active enzyme, one cannot confirm the substrate specificity, determine the subsite specificity, nor determine the kinetics or critical active site residues, all of which are essential for the design of inhibitors.

Memapsin 2 has been shown to be beta-secretase, a key protease involved in the production in human brain of beta-amyloid peptide from beta-amyloid precursor protein (for review, see Selkoe, D. J. Nature 399:23-31 (1999)). It is now generally accepted that the accumulation of beta-amyloid peptide in human brain is a major cause for Alzheimer's disease. Inhibitors specifically designed for human memapsin 2 should inhibit or decrease the formation of beta-amyloid peptide and the progression of the Alzheimer's disease.

Memapsin 2 belongs to the aspartic protease family. It is homologous in amino acid sequence to other eukaryotic aspartic proteases and contains motifs specific to that family. These structural similarities predict that memapsin 2 and other eukaryotic aspartic proteases share common catalytic mechanism, Davies, D. R., Annu. Rev. Biophys. Chem. 19, 189 (1990). The most successful inhibitors for aspartic proteases are mimics of the transition state of these enzymes. These inhibitors have substrate-like structure with the cleaved planar peptide bond between the carbonyl carbon and the amide nitrogen replaced by two tetrahedral atoms, such as hydroxyethylene [—CH(OH)—CH₂—], which was originally discovered in the structure of pepstatin (Marciniszyn et al., 1976).

A need exists to develop new, improved inhibitors of proteases involved in the production of beta-amyloid protein from beta-amyloid precursor protein, such as memapsin 2 inhibitors, that are effective, for example, in the treatment of Alzheimer's disease in humans.

### SUMMARY OF THE INVENTION

The present invention relates to inhibitors of memapsin 2 activity and methods of using the inhibitors of memapsin 2 to treat Alzheimer's disease in humans.

In one embodiment, the invention is an inhibitor of catalytically active memapsin 2 which binds to the active site of the memapsin 2 defined by the presence of two catalytic aspartic residues and substrate binding cleft, the inhibitor having an K, of less than or equal to 10⁻⁷ M.

In another embodiment, the invention includes a compound selected from the group consisting of MMI-005, MMI-012, MMI-017, MMI-018, MMI-025, MMI-026, MMI-037, MMI-039, MMI-040, MMI-065, MMI-066, MMI-070, and MMI-071.

In yet another embodiment, the invention includes a compound selected from the group consisting of MMI-012, MMI-017, MMI-018, MMI-026, MMI-037, MMI-039, MMI-040, MMI-070 and MMI-071.

Another embodiment includes a method for treating a patient to decrease the likelihood of developing or the progression of Alzheimer's disease comprising administering to the individual an effective amount of an inhibitor of memapsin 2 selected from the group consisting of MMI-005, MMI-012, MMI-017, MMI-018, MMI-025, MMI-026, MMI-037, MMI-039, MMI-040, MMI-065, MMI-070 and MMI-071.

In an additional embodiment, the invention is a method of determining the substrate side-chain preference in memapsin 2 sub-sites, comprising the steps of reacting a mixture of memapsin 2 substrates with memapsin 2 and determining the sub-site preference of memapsin 2 by determining relative initial hydrolysis rates of the mixture of memapsin 2 substrates.

In still another embodiment, the invention includes a method of determining the substrate side-chain preference in memapsin 2 sub-sites. A combinatorial library of memapsin 2 inhibitors wherein the inhibitors comprise a base sequence taken from OM99-2 is prepared. The library of inhibitors is probed with memapsin 2 wherein the memapsin 2 may bind one or a plurality of inhibitors to generate one or a plurality of bound memapsin 2 and the bound memapsin 2 is detected with an antibody raised to memapsin 2 and an alkaline phosphatase conjugated secondary antibody.

In a further embodiment, the invention includes a method of treating a human suffering from Alzheimer's disease comprising administering to the human an inhibitor of catalytically active memapsin 2 which binds to the active site of the memapsin 2 defined by the presence of two catalytic aspartic residues and substrate binding cleft, the inhibitor having an Kᵢ of less than or equal to 10⁻⁷ M.

In yet another embodiment, the invention relates to a method of treating a human suffering from Alzheimer's disease comprising administering to the human an inhibitor of catalytically active memapsin 2 which binds to the active site of the memapsin 2 defined by the presence of two catalytic aspartic residues and substrate binding cleft, the inhibitor having an Kᵢ of less than or equal to 10⁻⁷ M, wherein the inhibitor has a root mean square difference of less than or equal to 0.5 Å for the side chain and backbone atoms for amino acids 28-441 of SEQ ID NO: 2.

An additional embodiment of the invention is a method of treating a human suffering from Alzheimer's disease comprising administering to the human a compound selected from the group consisting of MMI-012, MMI-017, MMI-018, MMI-026, MMI-037, MMI-039, MMI-040, MMI-070 and MMI-071.

In yet another embodiment, the invention relates to the use of an inhibitor of catalytically active memapsin 2 which binds to the active site of the memapsin 2 defined by the presence of two catalytic aspartic residues and substrate binding cleft, the inhibitor having an Kᵢ of less than or equal to 10⁻⁷ M for the manufacture of a medicament for the treatment of Alzheimer's disease in a human.

In an additional embodiment, the invention relates to the use of an inhibitor of catalytically active memapsin 2 which binds to the active site of the memapsin 2 defined by the presence of two catalytic aspartic residues and substrate binding cleft, the inhibitor having an Kᵢ of less than or equal to 10⁻⁷ M, wherein the inhibitor has a root mean square difference of less than or equal to 0.5 Å for the side chain and backbone atoms for amino acids 18-379 of memapsin 2, for the manufacture of a medicament for the treatment of Alzheimer's disease in a human.

In still another embodiment, the invention relates to the use of a compound selected from the group consisting of MMI-005, MMI-012, MMI-017, MMJ-018, MMI-025, MMI-026, MMI-037, MMI-039, MMI-040, MMI-065, MMI-066, MMI-070 and MMI-071, for the manufacture of a medicament for the treatment of Alzheimer's disease in a human.

The substrate and subsite specificity of recombinant, catalytically active memapsin 2 was used to design substrate analogs of the natural memapsin 2 substrate that can inhibit the function of memapsin 2. Initially, X-ray crystallography of memapsin 2 bound to a substrate analog, OM99-2, was used to determine the three dimensional structure of the memapsin 2, as well as the importance of the various residues in binding. Substrate analogs were then designed based on peptide sequences shown to be related to the natural peptide substrates for memapsin 2 and the crystallographic structure. The substrate analogs contain at least one analog of an amide (peptide) bond that is not capable of being cleaved by memapsin 2.

The substrate and subsite specificity of the catalytically active memapsin 2 have been further determined by a method which determines the initial hydrolysis rate of the substrates using mass spectroscopy, for example, MALDI-TOF/MS (matrix assisted laser desorption/ionization-time of flight/mass spectroscopy). Alternatively, the subsite specificity of memapsin was further determined by probing a library of inhibitors with memapsin 2 and subsequently detecting the bound memapsin 2 with an antibody raised to memapsin 2 and an alkaline phosphatase conjugated secondary antibody. The substrate and subsite specificity information was used to design additional substrate analogs of the natural memapsin 2 substrate that can inhibit the function of memapsin 2.

Processes for the synthesis of substrate analogues including isosteres at the sites of the critical amino acid residues were developed. Substrate analogues, OM99-1, OM99-2 and more than seventy other substrate analogues were synthesized. OM99-2 is based on an octapeptide Glu-Val-Asn-Leu-Ala-Ala-Glu-Phe (SEQ ID NO: 28) with the Leu-Ala peptide bond substituted by a transition-state isostere hydroxyethylene group. The inhibition constant of OM99-2 is 1.6×10⁻⁹ M against recombinant pro-memapsin 2. The inhibition constants of MMI-005, MMI-012, MMI-017, MMI-018, MMI-025, MMI-026, MMI-037, MMI-039, MMI-040, MMI-066, MMI-070, and MMI-071 have inhibition constants in the range of 1.4-61.4×10⁻⁹ M against recombinant pro-memapsin 2.

Compositions that inhibit memapsin 2 aspartic protease activity can be small molecules, which readily pass across the blood brain barrier, are administered orally, and are not inactivated by intestinal enzymes. Furthermore, it is desirable that such compositions are relatively inexpensive to manufacture and preferentially inhibit memapsin 2 cleavage of beta-amyloid precursor protein.

This information can be used by those skilled in the art to design additional new inhibitors, using commercially available software programs and techniques familiar to those in organic chemistry and enzymology, to design new inhibitors. For example, the side chains of the inhibitors may be modified to produce stronger interactions (through hydrogen bonding, hydrophobic interaction, charge interaction and/or van der Waal interaction) in order to increase inhibition potency. Based on this type of information, the residues with minor interactions may be eliminated from the new inhibitor design to decrease the molecular weight of the inhibitor. The side chains with no structural hindrance from the enzyme may be cross-linked to lock in the effective inhibitor conformation. This type of structure also enables the design of peptide surrogates which may effectively fill the binding sites of memapsin 2 yet produce better pharmaceutical properties. The rational design and screening of compounds for inhibitors, and their characterization are provided in this invention. Compositions effective for inhibition of memapsin 2 include small molecule inhibitors, and inhibitors that are capable of crossing the blood brain barrier. Such inhibitors can interact with memapsin 2, or its substrate, to inhibit cleavage by memapsin 2.

The invention described herein provides compounds which inhibit memapsin 2 activity, in particular the aspartic protease activity of memapsin 2, which converts beta-amyloid precursor protein to beta-amyloid protein. The compounds of the invention can be used to treat Alzheimer's disease in humans.

### BRIEF DESCRIPTION OF THE DRAWINGS

FIG. 1 is a schematic representation of inhibitors for memapsin 2. “A” and “B” denote aliphatic linkages (saturated or partially unsaturated) of any number of carbons, between side chains in positions P₁ and P₃, for example, the amino acid side chains P₁ Leu and P₃ Val in these respective positions. Other structural elements of the inhibitor are omitted for clarity.

FIG. 2 is a schematic representation of inhibitors for memapsin 2. “A” denotes aliphatic linkages (saturated or partially unsaturated) of any number of carbons, between side chains in positions P₂ and P₄, for example, the amino acid side chains P₂ Asn and P₄ Glu in these respective positions. Other structural elements of the inhibitor are omitted for clarity.

FIG. 3 is a schematic of the design for the side chain at the P₁′ subsite for the new memapsin 2 inhibitors based on the current crystal structure. Arrows indicate possible interactions between memapsin 2 and inhibitor. Other structural elements of inhibitor are omitted for clarity.

FIG. 4 is a schematic representation of inhibitors for memapsin 2. “A” and “B” denote aliphatic linkages (saturated or partially unsaturated) between side chains in positions P₁ and the backbone atoms of P₃, for example, the amino acid side chains P₁ Leu in this position. Other structural elements of the inhibitor are omitted for clarity.

FIG. 5 shows the relative specificity of memapsin 2 for amino acid residues in positions P₁′-P₄′. Letters above bars indicate the native amino acid at that position. Catalytic efficiency is expressed relative to the native amino acid at each position.

FIG. 6 depicts the nucleotide sequence of human Memapsin 2 (SEQ ID NO: 1).

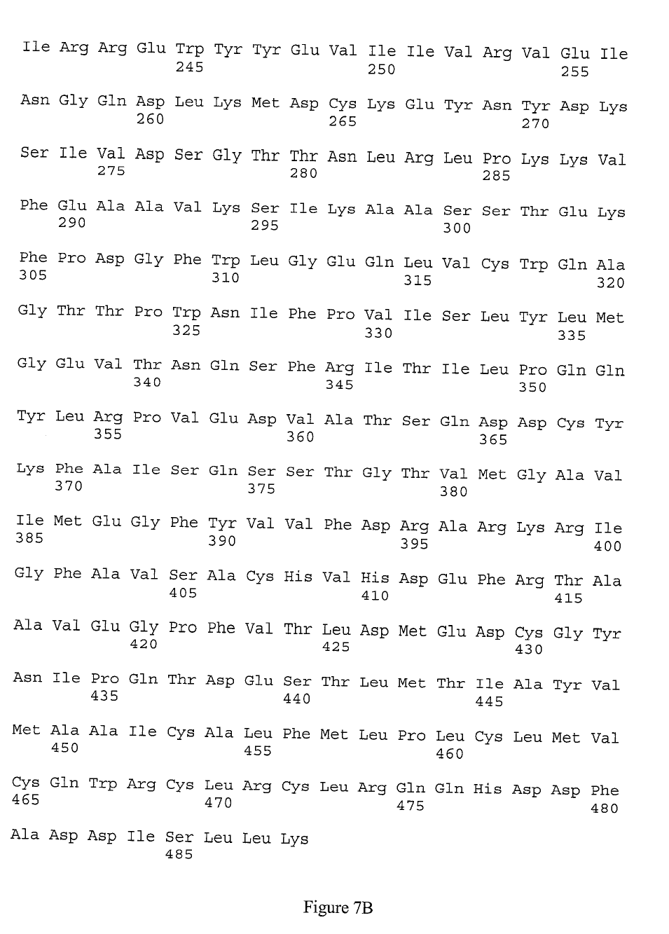

FIGS. 7A and 7B depict the partial protein sequence of human Memapsin 2, excluding the signal peptide (SEQ ID NO: 2). Amino acids 28-48 are remnant putative propeptide residues. Amino acids 58-61, 78, 80, 82-83, 116, 118-121, 156, 166, 174, 246, 274, 276, 278-281, 283, and 376-377 are residues in contact with the OM99-2 inhibitor. Amino acids 54-57, 61-68, 73-80, 86-89, 109-111, 113-118, 123-134, 143-154, 165-168, 198-202, and 220-224 are N-lobe beta strands. Amino acids 184-191 and 210-217 are N-lobe helices. Amino acids 237-240, 247-249, 251-256, 259-260, 273-275, 282-285, 316-318, 331-336, 342-348, 354-357, 366-370, 372-375, 380-383, 390-395, 400-405, and 418-420 are C-lobe beta strands. Amino acids 286-299, 307-310, 350-353, 384-387, and 427-431 are C-lobe helices.

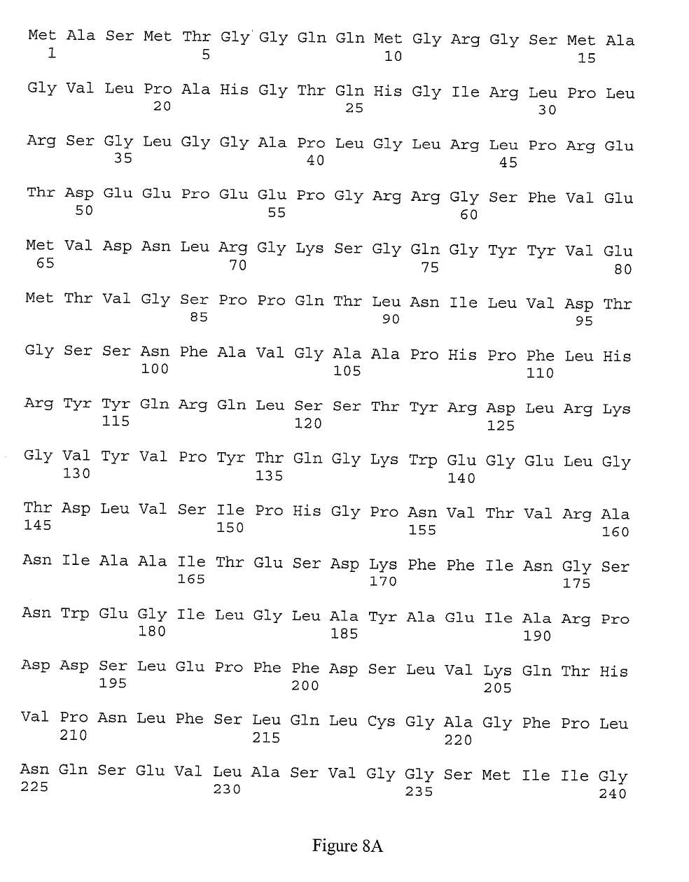

FIGS. 8A and 8B depict the protein sequence of human promemapsin 2 (SEQ ID NO: 3). Amino acids 1-15 are vector-derived residues. Amino acids 16-63 are a putative pro peptide. Amino acids 1-13 are derived from the T7 promoter. Amino acids 16-456 are Pro-memapsin 2-T1. Amino acids 16-421 are Promemapsin 2-T2.

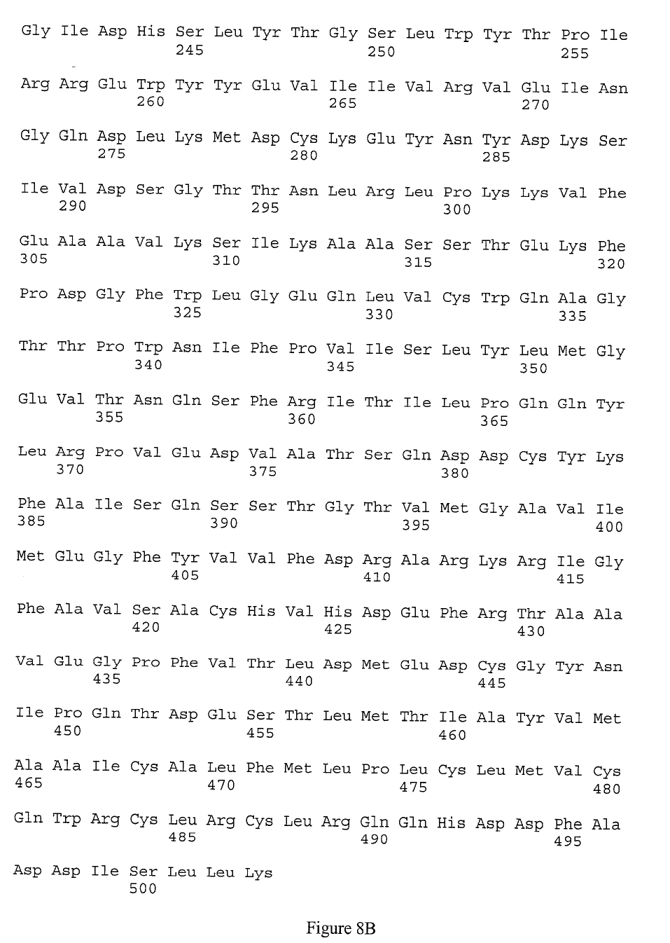

FIG. 9 depicts the amino acid sequence of human pre-promemapsin 2 (SEQ ID NO: 4). Amino acids 1-13 are the signal peptide. Amino acids 41-454 correspond to amino acids 28-441 of FIGS. 7A and 7B, and amino acids 43-456 of FIGS. 5A and 5B. The active site aspartic acids are at amino acid positions 93 and 289.

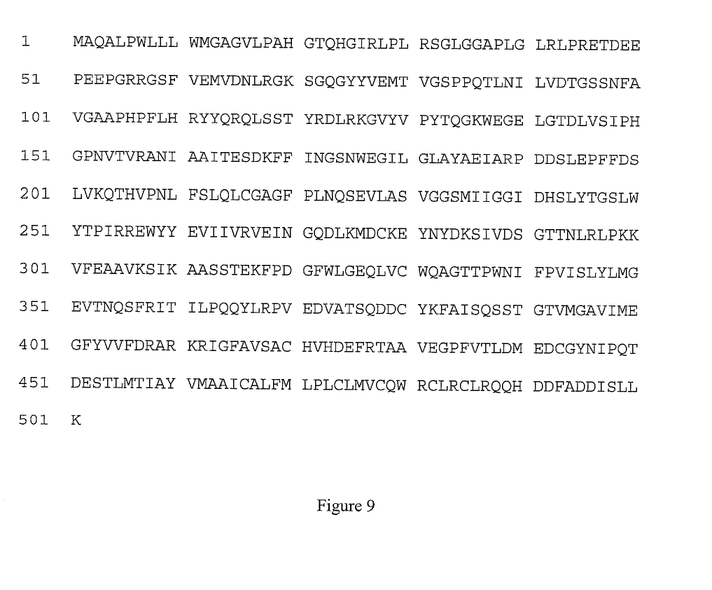

The foregoing and other objects, features and advantages of the invention will be apparent from the following more particular description of preferred embodiments of the invention.

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

I. Design and Synthesis of Inhibitors

Design of Substrate Analogs for Memapsin 2.

The five human aspartic proteases have homologous amino acid sequences and have similar three-dimensional structures. There are two aspartic residues in the active site and each residue is found within the signature aspartic protease sequence motif, Asp-Thr/Ser-Gly-. There are generally two homologous domains within an aspartic protease and the substrate binding site is positioned between these two domains, based on the three-dimensional structures. The substrate binding sites of aspartic proteases generally recognize eight amino acid residues. There are generally four residues on each side of the amide bond that is cleaved by the aspartic protease.

Typically the side chains of each amino acid are involved in the specificity of the substrate/aspartic protease interaction. The side chain of each substrate residue is recognized by regions of the enzyme which are collectively called sub-sites. The generally accepted nomenclature for the protease sub-sites and their corresponding substrate residues are shown below, where the double slash represents the position of bond cleavage.

| | | | | | | | | | |
|---|---|---|---|---|---|---|---|---|---|
| Protease sub-sites | S4 | S3 | S2 | S1 | | S1′ | S2′ | S3′ | S4′ |
| Substrate residues | P4 | P3 | P2 | P1 | // | P1′ | P2′ | P3′ | P4′ |

While there is a general motif for aspartic protease substrate recognition, each protease has a very different substrate specificity and breadth of specificity. Once the specificity of an aspartic protease is known, inhibitors can be designed based on that specificity, which interact with the aspartic protease in a way that prevents natural substrate from being efficiently cleaved. Some aspartic proteases have specificities that can accommodate many different residues in each of the sub-sites for successful hydrolysis. Pepsin and cathepsin D have this type of specificity and are said to have “broad” substrate specificity. When only a very few residues can be recognized at a sub-site, such as in renin, the aspartic protease is said to have a stringent or narrow specificity.

The information on the specificity of an aspartic protease can be used to design specific inhibitors in which the preferred residues are placed at specific sub-sites and the cleaved peptide bond is replaced by an analog of the transition-state. These analogs are called transition state isosteres. Aspartic proteases cleave amide bonds by a hydrolytic mechanism. This reaction mechanism involves the attack by a hydroxide ion on the β-carbon of the amino acid. Protonation must occur at the other atom attached to the β-carbon through the bond that is to be cleaved. If the β-carbon is insufficiently electrophilic or the atom attached to the bond to be cleaved is insufficiently nucleophilic, the bond will not be cleaved by a hydrolytic mechanism. Analogs exist which do not mimic the transition state but which are non-hydrolyzable, but transition state isosteres mimic the transition state specifically and are non-hydrolyzable.

Transition state theory indicates that it is the transition state intermediate of the reaction which the enzyme catalyzes for which the enzyme has its highest affinity. It is the transition state structure, not the ground state structure, of the substrate which will have the highest affinity for its given enzyme. The transition state for the hydrolysis of an amide bond is tetrahedral while the ground state structure is planar. A typical transition-state isostere of aspartic protease is —CH(OH)—CH₂—, as was first discovered in pepstatin by Marciniszyn et al. (1976). The transition-state analogue principles have been successfully applied to inhibitor drugs for human immunodeficiency virus protease, an aspartic protease. Many of these are currently in clinical use. Information on the structure, specificity, and types of inhibitors can be found in Tang, Acid Proteases, Structure, Function and Biology, Adv. in Exptl. Med. Biol. vol. 95 (Plenum Press, NY 1977); Kostka, Aspartic Proteinases and their Inhibitors (Walter de Gruyter, Berlin 1985); Dunn, Structure and Functions of the Aspartic Proteinases, Adv. in Exptl. Med. Biol. 306 (Plenum Press, NY 1991); Takahashi, Aspartic Proteases, Structure, Function, Biology, Biomedical Implications, Adv. in Exptl. Med. Biol. 362 (Plenum Press, NY 1995); and James, Aspartic Proteinases, Retroviral and Cellular Enzymes, Adv. in Exptl. Med. Biol. 436 (Plenum Press, NY 1998), the teachings of all of which are incorporated herein in their entirety).

Substrate analog compositions are generally of the general formula X-L₄-P₄-L₃-P₃-L₂-P₂-L₁-P₁-L₀-P₁′-L₁′-P₂′-L₂′-P₃′-L₃′-P₄′-L₄′-Y. The substrate analog compositions are analogs of small peptide molecules. Their basic structure is derived from peptide sequences that were determined through structure/function studies. It is understood that positions represented by Pₓ represent the substrate specificity position relative to the cleavage site which is represented by an -L₀-. The positions of the compositions represented by Lₓ represent the linking regions between each substrate specificity position, Pₓ.

In a natural substrate for memapsin 2, a Pₓ-Lₓ pair would represent a single amino acid of the peptide which is to be cleaved. In the present general formula, each Pₓ part of the formula refers to the α-carbon and side chain functional group of each would be amino acid. Thus, the Pₓ portion of an PₓLₓ pair for alanine represents HC—CH₃. The general formula representing the Pₓ portion of the general composition is —R₁CR₃—.

In general R₁ can be either CH₃ (side chain of alanine), CH(CH₃)₂ (side chain of valine), CH₂CH(CH₃)₂ (side chain of leucine), (CH₃)CH(CH₂CH₃) (side chain of isoleucine), CH₂(indole) (side chain of tryptophan), CH₂(benzene) (side chain of phenylalanine), CH₂CH₂SCH₃ (side chain of methionine), H (side chain of glycine), CH₂OH (side chain of serine), CHOHCH₃ (side chain of threonine), CH₂(phenol) (side chain of tyrosine), CH₂SH (side chain of cysteine), CH₂CH₂CONH₂ (side chain of glutamine), CH₂CONH₂ (side chain of asparagine), CH₂CH₂CH₂CH₂NH₂ (side chain of lysine), CH₂CH₂CH₂NHC(NH)(NH₂) (side chain of arginine), CH₂(imidazole) (side chain of histidine), CH₂COOH (side chain of aspartic acid), CH₂CH₂COOH (side chain of glutamic acid), and functional natural and non-natural derivatives or synthetic substitutions of these.

It is most preferred that R₃ is a single H. In general, however, R₃ can be alkenyl, alkynal, alkenyloxy, and alkynyloxy groups that allow binding to memapsin 2. Preferably, alkenyl, alkynyl, alkenyloxy and alkynyloxy groups have from 2 to 40 carbons, and more preferably from 2 to 20 carbons, from 2 to 10 carbons, or from 2 to 3 carbons, and functional natural and non-natural derivatives or synthetic substitutions of these.

The Lₓ portion of the Pₓ-Lₓ pair represents the atoms linking the Pₓ regions together. In a natural substrate the Lₓ represents the β-carbon attached to the amino portion of what would be the next amino acid in the chain. Thus, Lₓ would be represented by —CO—NH—. The general formula for Lₓ is represented by R₂. In general R₂ can be CO—HN (amide), CH(OH)(CH₂) (hydroxyethylene), CH(OH)CH(OH) (dihydroxyethylene), CH(OH)CH₂NH (hydroxyethylamine), PO(OH)CH₂ (phosphinate), CH₂NH (reduced amide). It is understood that more than one L— maybe an isostere as long as the substrate analog functions to inhibit aspartic protease function.

Ls which are not isosteres may either be an amide bond or mimetic of an amide bond that is non-hydrolyzable.

X and Y represent molecules which are not typically involved in the recognition by the aspartic protease recognition site, but which do not interfere with recognition. It is preferred that these molecules confer resistance to the degradation of the substrate analog. Preferred examples would be amino acids coupled to the substrate analog through a non-hydrolyzable bond. Other preferred compounds are capping agents. Still other preferred compounds are compounds that could be used in the purification of the substrate analogs such as biotin.

As used herein, alkyl refers to substituted or unsubstituted straight, branched or cyclic alkyl groups; and alkoxyl refers to substituted or unsubstituted straight, branched or cyclic alkoxy. Preferably, alkyl and alkoxy groups have from 1 to 40 carbons, and more preferably from 1 to 20 carbons, from 1 to 10 carbons, or from 1 to 3 carbons.

As used herein, alkenyl refers to substituted or unsubstituted straight chain or branched alkenyl groups; alkynyl refers to substituted or unsubstituted straight chain or branched alkynyl groups; alkenyloxy refers to substituted or unsubstituted straight chain or branched alkenyloxy; and alkynyloxy refers to substituted or unsubstituted straight chain or branched alkynyloxy. Preferably, alkenyl, alkynyl, alkenyloxy and alkynyloxy groups have from 2 to 40 carbons, and more preferably from 2 to 20 carbons, from 2 to 10 carbons, or from 2 to 3 carbons.

As used herein, alkaryl refers to an alkyl group that has an aryl substituent; aralkyl refers to an aryl group that has an alkyl substituent; heterocyclic-alkyl refers to a heterocyclic group with an alkyl substituent; alkyl-heterocyclic refers to an alkyl group that has a heterocyclic substituent.

The substituents for alkyl, alkenyl, alkynyl, alkoxy, alkenyloxy, and alkynyloxy groups can be halogen, cyano, amino, thio, carboxy, ester, ether, thioether, carboxamide, hydroxy, or mercapto. Further, the groups can optionally have one or more methylene groups replaced with a heteroatom, such as O, NH or S.

A number of different substrates were tested and analyzed, and the cleavage rules for Memapsin 2 were determined. The results of the substrates which were analyzed are presented in Table 1 and the rules determined from these results are summarized below.

(1) The primary specificity site for a memapsin 2 substrate is subsite position, S₁′. This means that the most important determinant for substrate specificity in memapsin 2 is the amino acid, P1′. P₁′ may be a small side chain for memapsin 2 to recognize the substrate. Preferred embodiments are substrate analogs where R₁ of the P₁′ position is either H (side chain of glycine), CH₃ (side chain of alanine), CH₂OH (side chain of serine), or CH₂COOH (side chain of aspartic acid). Embodiments that have an R₁ structurally smaller than CH₃ (side chain of alanine) or CH₂OH (side chain of serine) are also preferred. However, substrates in Table 1 nay not, due to their amino acid composition, provide a complete representation of the substrate specificity of memapsin 2. Therefore, P₁′ may not be limited to the small residues mentioned, but may also include the following:

CH₂CH(CH₃)₂ (sidechain of leucine),

(CH₃)CH(CH₂CH₃) (sidechain of isoleucine),

CH₂(INDOLE) (sidechain of tryptophan),

CH₂(BENZNE) (sidechain of phenylalanine),

CH(CH₃)₂ (sidechain of valine),

CH₂(PHENOL) (sidechain of tyrosine),

CH₂CH₂SCH₃ (sidechain of methionine),

CH(CH₃OH)(sidechain of threonine),

CH₂CONH (sidechain of asparagine),

CH₂CH₂CONH (sidechain of glutamine),

CH₂CH₂COOH (sidechain of glutamic acid),

CH₂CH₂CH₂CH₂NH₃(sidechain of lysine),

CH₂CH₂CH₂NC(NH₂)₂ (sidechain of arginine),

CH₂(IMIDAZOLE) (sidechain of histidine).

(2) There are no specific sequence requirements at positions P₄ P₃, P₂, P₁, P₂′, P₃′, and P₄′. Each site can accommodate any other amino acid residue in singularity as long as rule number 3 is met.

(3) At least two of the remaining seven positions, P₄, P₃, P₂, P₁, P₂′, P₃′, and P₄′, must have an R₁ which is made up of a hydrophobic residue. It is preferred that there are at least three hydrophobic residues in the remaining seven positions, P₄, P₃, P₂, P₁, P₂′, P₃′, and P₄′. Preferred R₁ groups for the positions that contain a hydrophobic group are CH₃ (side chain of alanine), CH(CH₃)₂ (side chain of valine), CH₂CH(CH₃)₂ (side chain of leucine), (CH₃)CH(CH₂ CH₃) (side chain of isoleucine), CH₂(indole) (side chain of tryptophan), CH₂(benzene) (side chain of phenylalanine), CH₂CH₂SCH₃ (side chain of methionine) CH₂(phenol) (side chain of tyrosine). It is more preferred that the hydrophobic group be a large hydrophobic group. Preferred R₁s which contain large hydrophobic groups are CH(CH₃)₂ (side chain of valine), CH₂CH(CH₃)₂ (side chain of leucine), (CH₃)CH(CH₂ CH₃) (side chain of isoleucine), CH₂(indole) (side chain of tryptophan), CH₂(benzene) (side chain of phenylalanine), CH₂CH₂SCH₃ (side chain of methionine) CH₂(phenol) (side chain of tyrosine). It is most preferred that positions with a hydrophobic R₁ are CH(CH₃)₂ (side chain of valine), CH₂CH(CH₃)₂ (side chain of leucine), CH₂(benzene) (side chain of phenylalanine), CH₂CH₂SCH₃ (side chain of methionine), or CH₂(phenol) (side chain of tyrosine).

(4) None of the eight positions, P₄, P₃, P₂, P₁, P₁,′ P₂′, P₃′, and P₄′ may have a proline side chain at its R1 position.

(5) Not all subsites must have an P represented in the analog. For example, a substrate analog could have X-P₂-L₁-P₁-L₀-P₁′-L₁′-P₂′-L₂′-P₃′-L₃′-Y or it could have X-L₁-P₁-L₀-P₁′-L₁′-P₂′-L₂′-P₃′-L₃′-P₄′-L₄′-Y.

Preferred substrate analogs are analogs having the sequences disclosed in Table 1, with the non-hydrolyzable analog between P1 and P1′.

Combinatorial Chemistry to Make Inhibitors

Combinatorial chemistry includes but is not limited to all methods for isolating molecules that are capable of binding either a small molecule or another macromolecule. Proteins, oligonucleotides, and polysaccharides are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992, the teachings of which are incorporated herein in their entirety). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992, the teachings of all of which are incorporated herein in their entirety).

Techniques aimed at similar goals exist for small organic molecules, proteins and peptides and other molecules known to those of skill in the art. Screening sets of molecules for a desired activity whether based on libraries of small synthetic molecules, oligonucleotides, proteins or peptides is broadly referred to as combinatorial chemistry.

There are a number of methods for isolating proteins either have de novo activity or a modifed activity. For example, phage display libraries have been used for a number of years. A preferred method for isolating proteins that have a given function is described by Roberts and Szostak (Roberts R. W. and Szostak J. W. Proc. Natl. Acad. Sci. USA, 94 (23)12997-302 (1997), the teachings of which are incorporated herein in their entirety). Another preferred method for combinatorial methods designed to isolate peptides is described in Cohen et al. (Cohen B. A., et al., Proc. Natl. Acad. Sci. USA 95 (24):14272-7 (1998), the teachings of which are incorporated herein in their entirety). This method utilizes a modified two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989), the teachings of which are incorporated herein in their entirety). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attach to an acidic activation domain. A peptide of choice, for example an extracellular portion of memapsin 2 is attached to a DNA binding domain of a transcriptional activation protein, such as Gal 4. By performing the Two-hybrid technique on this type of system, molecules that bind the extracellular portion of memapsin 2 can be identified.

Screening of Small Molecule Libraries

In addition to these more specialized techniques, methodology well known to those of skill in the art, in combination with various small molecule or combinatorial libraries, can be used to isolate and characterize those molecules which bind to or interact with the desired target, either memapsin 2 or its substrate. The relative binding affinity of these compounds can be compared and optimum inhibitors identified using competitive or non-competitive binding studies which are well known to those of skill in the art. Preferred competitive inhibitors are non-hydrolyzable analogs of memapsin 2. Another will cause allosteric rearrangements which prevent memapsin 2 from functioning or folding correctly.

Computer assisted Rational Drug Design

Another way to isolate inhibitors is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from X-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. For example, using NMR spectroscopy, Inouye and coworkers were able to obtain the structural information of N-terminal truncated TSHK (transmembrane sensor histidine kinases) fragments which retain the structure of the individual sub-domains of the catalytic site of a TSHK. On the basis of the NMR study, they were able to identify potential TSHK inhibitors (U.S. Pat. No. 6,077,682 to Inouye, the teachings of which are incorporated herein in their entirety). Another good example is based on the three-dimensional structure of a calcineurin/FKBP12/FK506 complex determined using high resolution X-ray crystallography to obtain the shape and structure of both the calcineurin active site binding pocket and the auxiliary FKBP12/FK506 binding pocket (U.S. Pat. No. 5,978,740 to Armistead, the teachings of which are incorporated herein in their entirety). With this information in hand, researchers can have a good understanding of the association of natural ligands or substrates with the binding pockets of their corresponding receptors or enzymes and are thus able to design and make effective inhibitors.

Prediction of molecule-compound interaction when small changes are made in one or both requires molecular mechanics software and computationally intensive computers, usually coupled with user-friendly, menu-driven interfaces between the molecular design program and the user. Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxicol. 29, 111-122; Perry and Davies, QSAR: Quantitative Structure-Activity Relationships in Drug Design pp. 189-193 (Alan R. Liss, Inc. 1989; Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090, the teachings of all of which are incorporated herein in their entirety). Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario.

Although described above with reference to design and generation of compounds which could alter binding, one could also screen libraries of known compounds, including natural products or synthetic chemicals, and biologically active materials, including proteins, for compounds which alter substrate binding or enzymatic activity.

Screening of Libraries

Design of substrate analogs and rational drug design are based on knowledge of the active site and target, and utilize computer software programs that create detailed structures of the enzyme and its substrate, as well as ways they interact, alone or in the presence of inhibitor. These techniques are significantly enhanced with X-ray crystallographic data in hand. Inhibitors can also be obtained by screening libraries of existing compounds for those which inhibit the catalytically active enzyme. In contrast to reports in the literature relating to memapsin 2, the enzyme described herein has activity analogous to the naturally produced enzyme, providing a means for identifying compounds which inhibit the endogenous activity. These potential inhibitors are typically identified using high throughput assays, in which enzyme, substrate (preferably a chromogenic substrate) and potential inhibitor (usually screened across a range of concentrations) are mixed and the extent of cleavage of substrate determined. Potentially useful inhibitors are those which decrease the amount of cleavage.

II. Preparation of Catalytically Active Recombinant Memapsin 2

Cloning and Expression of Memapsin 2

Memapsin 2 was cloned and the nucleotide (SEQ ID NO: 1) and predicted amino acid (SEQ ID NO: 2) sequences were determined, as described in Example 1. The cDNA was assembled from the fragments. The nucleotide and the deduced protein sequence are shown in SEQ ID NOS: 1 and 2, respectively. The protein is the same as the aspartic proteinase 2 (ASP2) described in EP 0 855 444 A by SmithKline Beecham Pharmaceuticals, (published Jul. 29, 1998), U.S. Pat. No. 6,319,689, Sinha, et al., Nature 402, 537-540 (December 1999) and Vassar, et al., Science 286, 735-741 (Oct. 22, 1999), the teachings of all of which are incorporated herein in their entirety).

Pro-memapsin 2 is homologous to other aspartic proteases, and shares homology with mouse ASP1 (U.S. Pat. No. 6,291,223, the teachings of which are incorporated herein in its entirety). Based on the alignments, Pro-memapsin 2 contains a pro region, an aspartic protease region, and a trans-membrane region near the C-terminus. The C-terminal domain is over 80 residues long. The active enzyme is memapsin 2 and its pro-enzyme is pro-memapsin 2.

Refolding Catalytically Active Enzyme

In order to determine the substrate specificity and to design inhibitors, it is necessary to express catalytically active recombinant enzyme. Since the active site is not in the transmembrane region and activity does not require membrane anchoring, memapsin 2 was expressed in E. coli in two different lengths, both without the transmembrane region, and purified, as described in Example 3. The procedures for the culture of transfected bacteria, induction of synthesis of recombinant proteins and the recovery and washing of inclusion bodies containing recombinant proteins are essentially as described by Lin et al, (1994), the teachings of which are incorporated herein in its entirety. For refolding, the protein is dissolved in a strong denaturing/reducing solution such as 8 M urea/100 mM beta-mercaptoethanol. The rate at which the protein is refolded, and in what solution, is critical to activity. In one method, the protein is dissolved into 8 M urea/100 mM beta-mercaptoethanol then rapidly diluted into 20 volumes of 20 mM-Tris, pH 9.0, which is then slowly adjusted to pH 8 with 1 M HCl. The refolding solution is then kept at 4° C. for 24 to 48 hours before proceeding with purification. In the second method, an equal volume of 20 mM Tris, 0.5 mM oxidized/1.25 mM reduced glutathione, pH 9.0 is added to rapidly stirred pro-memapsin 2 in 8 M urea/10 mM beta-mercaptoethanol. The process is repeated three more times with 1 hour intervals. The resulting solution is then dialyzed against sufficient volume of 20 mM Tris base so that the final urea concentration is 0.4 M. The pH of the solution is then slowly adjusted to 8.0 with 1 M HCl.

The refolded protein is then further purified by column chromatography, based on molecular weight exclusion, and/or elution using a salt gradient, and analyzed by SDS-PAGE analysis under reduced and non-reduced conditions.

III. Substrate Specificity and Enzyme Kinetics of Memapsin 2

The inhibitors can be screened for inhibition of binding and cleavage by memapsin 2 of its substrate.

Substrate Specificity

The presence of memapsin 2 (M2) in the brain indicated that it might hydrolyze the P-amyloid precursor protein (APP). As described below, detailed enzymatic and cellular studies demonstrated that M2 fits all the criteria of the β-secretase. The M2 three-dimensional structure modeled as a type I integral membrane protein. The model suggested that its globular protease unit can hydrolyze a membrane anchored polypeptide at a distance range of 20-30 residues from the membrane surface. As a transmembrane protein of the brain, APP is a potential substrate and its beta-secretase site, located about 28 residues from the plasma membrane surface, is within in the range for M2 proteolysis.

A synthetic peptide derived from this site (SEVKM/DAEFR) (SEQ ID NO: 5) was hydrolyzed by M2_{pd} (modified M2 containing amino acids from Ala^{-8P} to Ala³²⁶) at the beta-secretase site (marked by /). A second peptide (SEVNL/DAEFR) (SEQ ID NO: 6) derived from the APP beta-secretase site and containing the ‘Swedish mutation’ (Mullan, M. et al., Nature Genet. 2:340-342 (1992), the teachings of which are incorporated herein in its entirety), known to elevate the level of Aβ production in cells (Citron, M. et al., Nature 260:672-674 (1992), the teachings of which are incorporated herein in its entirety), was hydrolyzed by M2_{pd} with much higher catalytic efficiency. Both substrates were optimally cleaved at pH 4.0. A peptide derived from the processing site of presenilin 1 (SVNM/AEGD) (SEQ ID NO: 7) was also cleaved by M2_{pd} with less efficient kinetic parameters. A peptide derived from the APP gamma-secretase site (KGGVVIATVIVK) (SEQ ID NO: 8) was not cleaved by M2_{pd}. Pepstatin A inhibited M2_{pd} poorly (IC₅₀ approximately approximately 0.3 mM). The kinetic parameters indicate that both presenilin 1 (k_{cat}, 0.67 s⁻¹; Kₘ, 15.2 mM; k_{cat}/Kₘ, 43.8 s⁻¹M⁻¹) and native APP peptides (k_{cat}/Kₘ, 39.9 s⁻¹M⁻¹) are not as good substrates as the Swedish APP peptide (k_{cat}, 2.45 s⁻¹,Kₘ, 1 mM; k_{cat}/Kₘ, 2450 s⁻¹M⁻¹).

To determine if M2 possesses an APP beta-secretase function in mammalian cells, memapsin 2 was transiently expressed in HeLa cells (Lin, X., et al., FASEB J. 7:1070-1080 (1993), the teachings of which are incorporated herein in its entirety), metabolically pulse-labeled with ³⁵S-Met, then immunoprecipitated with anti-APP antibodies for visualization of APP-generated fragments after SDS-polyacrylamide electrophoresis and imaging. SDS-PAGE patterns of immuno-precipitated APP Nβ-fragment (97 kD band) from the conditioned media (2 h) of pulse-chase experiments showed that APP was cleaved by M2. Controls transfected with APP alone and co-transfected with APP and M2 with Bafilomycin A1 added were performed. SDS-PAGE patterns of APP βC-fragment (12 kD) were immunoprecipitated from the conditioned media of the same experiment as discussed above. Controls transfected with APP alone; co-transfected with APP and M2; co-transfected with APP and M2 with Bafilomycin A1; transfections of Swedish APP; and co-transfections of Swedish APP and M2 were performed. SDS-PAGE gels were also run of immuno-precipitated M2 (70 kD), M2 transfected cells; untransfected HeLa cells after long time film exposure; and endogenous M2 from HEK 293 cells. SDS-PAGE patterns of APP fragments (100 kD betaN-fragment and 95 kD betaN-fragment) recovered from conditioned media after immuno-precipitation using antibodies specific for different APP regions indicated that memapsin 2 cleaved APP.

Cells expressing both APP and M2 produced the 97 kD APP beta N-fragment (from the N-terminus to the beta-secretase site) in the conditioned media and the 12 kD betaC-fragment (from the beta-secretase site to the C-terminus) in the cell lystate. Controls transfected with APP alone produced little detectable betaN-fragment and no beta C-fragment. Bafilomycin A1, which is known to raise the intra-vesicle pH of lysosomes/endosomes and has been shown to inhibit APP cleavage by beta-secretase (Knops, J. et al., J. Biol. Chem. 270: 2419-2422 (1995), the teachings of which are incorporated herein in its entirety), abolished the production of both APP fragments beta N- and beta C- in co-transfected cells. Cells transfected with Swedish APP alone did not produce the beta C-fragment band in the cell lysate but the co-transfection of Swedish APP and M2 did. This Swedish beta C-fragment band is more intense than that of wild-type APP. A 97-kD beta N-band is also seen in the conditioned media but is about equal intensity as the wild-type APP transfection.

These results indicate that M2 processes the beta-secretase site of APP in acidic compartments such as the endosomes. To establish the expression of transfected M2 gene, the pulse-labeled cells were lysed and immuno-precipitated by anti-M2 antibodies. A 70 kD M2 band was seen in cells transfected with M2 gene, which has the same mobility as the major band from HEK 293 cells known to express beta-secretase (Citron, M. et al, Nature 260:672-674 (1992), the teachings of which are incorporated herein in its entirety). A very faint band of M2 is also seen, after a long film exposure, in untransfected HeLa cells, indicating a very low level of endogenous M2, which is insufficient to produce betaN- or betaC-fragments without M2 transfection. Antibody Aβ₁₋₁₇, which specifically recognizes residues 1-17 in Aβ peptide, was used to confirm the correct beta-secretase site cleavage. In cells transfected with APP and M2, both beta N- and beta N-fragments are visible using an antibody recognizing the N-terminal region of APP present in both fragments. Antibody Aβ₁₋₁₇ recognize the beta N-fragment produced by endogenous beta-secretase in the untransfected cells. This antibody was, however, unable to recognize the betaN-fragment known to be present in cells co-transfected with APP and M2. These observations confirmed that betaN-fragment is the product of beta-secretase site cut by M2, which abolished the recognition epitope of Aβ₁₋₁₇.

In specificity studies, it was found that M2_{pd} cleaved its pro peptide (2 sites) and the protease portion (2 sites) during a 16 h incubation after activation (Table 1). Besides the three peptides discussed above, M2_{pd} also cleaved oxidized bovine insulin B chain and a synthetic peptide Nch. Native proteins were not cleaved by M2_{pd}.

These same methods can be used in combination with the disclosed inhibitors to screen for efficacy in inhibiting the memapsin.

IV. Methods of diagnosis and treatment

Inhibitors can be used in the diagnosis and treatment and/or prevention of Alzheimer's disease and conditions associated therewith, such as elevated levels of the forty-two amino acid peptide cleavage product, and the accumulation of the peptide in amyeloid plaques.

Diagnostic Uses

The substrate analogs can be used as reagents for specifically binding to memapsin 2 or memapsin 2 analogs and for aiding in memapsin 2 isolation and purification or characterization, as described in the examples. The inhibitors and purified recombinant enzyme can be used in screens for those individuals more genetically prone to develop Alzheimer's disease.

Therapeutic Uses

Recombinant human memapsin 2 cleaves a substrate with the sequence LVNM/AEGD (SEQ ID NO: 9). This sequence is the in vivo processing site sequence of human presenilins. Both presenilin 1 and presenilin 2 are integral membrane proteins. They are processed by protease cleavage, which removes the N terminal sequence from the unprocessed form. Once processed, presenilin forms a two-chain heterodimer (Capell et al., J. Biol. Chem. 273, 3205 (1998); Thinakaran et al., Neurobiol. Dis. 4, 438 (1998); Yu et al, Neurosci Lett. 2;254(3): 125-8 (1998), the teachings of all of which are incorporated herein in their entirety), which is stable relative to the unprocessed presenilins. Unprocessed presenilines are quickly degraded (Thinakaran et al., J. Biol. Chem. 272, 28415 (1997); Steiner et al., J. Biol. Chem. 273, 32322 (1998), the teachings of all of which are incorporated herein in their entirety). It is known that presenilin controls the in vivo activity of beta-secretase, which in turn cleaves the amyloid precursor protein (APP) leading to the formation of Aβ42. The accumulation of Aβ42 in the brain cells is known to be a major cause of Alzheimer's disease (for review, see Selkoe, 1998, the teachings of which are incorporated herein in its entirety). The activity of presenilin therefore enhances the progression of Alzheimer's disease. This is supported by the observation that in the absence of presenilin gene, the production of Aβ42 peptide is lowered (De Strooper et al., Nature 391, 387 (1998), the teachings of which are incorporated herein in its entirety). Since unprocessed presenilin is degraded quickly, the processed, heterodimeric presenilin must be responsible for the accumulation of Aβ42 leading to Alzheimer's disease. The processing of presenilin by memapsin 2 would enhance the production of Aβ42 and therefore, further the progress of Alzheimer's disease. Therefore a memapsin 2 inhibitor that crosses the blood brain barrier can be used to decrease the likelihood of developing or slow the progression of Alzheimer's disease which is mediated by deposition of Aβ42. Since memapsin 2 cleaves APP at the beta cleavage site, prevention of APP cleavage at the beta cleavage site will prevent the build up of Aβ42.

Pharmaceutically Acceptable Carriers

The inhibitors will typically be administered orally or by injection. Oral administration is preferred. Alternatively, other formulations can be used for delivery by pulmonary, mucosal or transdermal routes. The inhibitor will usually be administered in combination with a pharmaceutically acceptable carrier. Pharmaceutical carriers are known to those skilled in the art. The appropriate carrier will typically be selected based on the mode of administration. Pharmaceutical compositions may also include one or more active ingredients such as antimicrobial agents, antiinflammatory agents, and analgesics.

Preparations for parenteral administration or administration by injection include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Preferred parenteral vehicles include sodium chloride solution, Ringer's dextrose, dextrose and sodium chloride, lactated Ringer's, or fixed oils. Intravenous vehicles include fluid and nutrient replenishers, and electrolyte replenishers (such as those based on Ringer's dextrose).

Formulations for topical (including application to a mucosal surface, including the mouth, pulmonary, nasal, vaginal or rectal) administration may include ointments, lotions, creams, gels, drops, suppositories, sprays, liquids and powders. Formulations for these applications are known. For example, a number of pulmonary formulations have been developed, typically using spray drying to formulate a powder having particles with an aerodynanmic diameter of between one and three microns, consisting of drug or drug in combination with polymer and/or surfactant.

Compositions for oral administration include powders or granules, suspensions or solutions in water or non-aqueous media, capsules, sachets, or tablets. Thickeners, flavorings, diluents, emulsifiers, dispersing aids or binders may be desirable.

Peptides as described herein can also be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Dosages

Dosing is dependent on severity and responsiveness of the condition to be treated, but will normally be one or more doses per day, with course of treatment lasting from several days to several months or until the attending physician determines no further benefit it if will be obtained. Persons of ordinary skill can determine optimum dosages, dosing methodologies and repetition rates.

The dosage ranges are those large enough to produce the desired effect in which the symptoms of the memapsin 2 mediated disorder are alleviated (typically characterized by a decrease in size and/or number of amyloid plaque, or by a failure to increase in size or quantity), or in which cleavage of the Aβ42 peptide is decreased. The dosage can be adjusted by the individual physician in the event of any counterindications.

The present invention will be further illustrated by the following examples which are not intended to be limiting in any way.

Exemplification

### EXAMPLE 1

### Proteolytic Activity and Cleavage-Site Preferences of Recombinant Memapsin 2

The amino acid sequence around the proteolytic cleavage sites of APP was determined in order to establish the specificity of memapsin 2. Recombinant pro-memapsin 2-T1 was incubated in 0.1 M sodium acetate, pH 4.0, for 16 hours at room temperature in order to create autocatalyzed cleavages. The products were analyzed using SDS-polyacrylamide gel electrophoresis. Several bands which corresponded to molecular weights smaller than that of pro-memapsin 2 were observed. The electrophoretic bands were trans-blotted onto a PVDF membrane. Four bands were chosen and subjected to N-terminal sequence determination in a Protein Sequencer. The N-terminal sequence of these bands established the positions of proteolytic cleavage sites on pro-memapsin 2.

In addition, the oxidized U-chain of bovine insulin and two different synthetic peptides were used as substrates for memapsin 2 to determine the extent of other hydrolysis sites. These reactions were carried out by auto-activated pro-memapsin 2 in 0.1 M sodium acetate, pH 4.0, which was then incubated with the peptides. The hydrolytic products were subjected to HPLC on a reversed phase C-18 column and the eluent peaks were subjected to electrospray mass spectrometry for the determination of the molecular weight of the fragments. Two hydrolytic sites were identified on oxidized insulin B-chain (Table1). Three hydrolytic sites were identified from peptide NCH-gamma. A single cleavage site was observed in synthetic peptide PS1-gamma, whose sequence (LVNMAEGD) (SEQ ID NO: 10) is derived from the beta-processing site of human presenilin 1 (Table 1).

TABLE 1
Substrate Specificity of Memapsin 2

| Site | | | | | | | | | | |
|---|---|---|---|---|---|---|---|---|---|---|
| # | Substrate | P4 | P3 | P2 | P1 | P1′ | P2′ | P3′ | P4′ | |
| 1 | Pro- | R | G | S | M | A | G | V | L | SEQ ID NO: 11 |
| | memapsin 2 | | | | | | | | | (aa 12-18 of SEQ ID |
| | | | | | | | | | | NO: 3) |
| 2 | | G | T | Q | H | G | I | R | L | SEQ ID NO: 12 |
| | | | | | | | | | | (aa 23-30 of SEQ ID |
| | | | | | | | | | | NO: 3) |
| 3 | | S | S | N | F | A | V | G | A | SEQ ID NO: 13 |
| | | | | | | | | | | (aa 98-105 of SEQ |
| | | | | | | | | | | ID NO: 3) |
| 4 | | G | L | A | Y | A | E | I | A | SEQ ID NO: 14 |
| | | | | | | | | | | (aa 183-190 of SEQ |
| | | | | | | | | | | ID NO: 3) |
| 5 | Oxidized | H | L | C\hat{ } | G | S | H | L | V | SEQ ID NO: 15 |
| 6 | Insulin B- | C\hat{ } | G | E | R | G | F | F | Y | C\hat{ } is cysteic acid; |
| | Chain′ | | | | | | | | | SEQ ID NO: 16 |
| | | | | | | | | | | C\hat{ } is cysteic acid |
| 7 | Synthetic | | | | V | G | S | G | V | Three sites cleaved |
| 8 | Peptide* | | V | G | S | G | V | L | L | in a peptide: |
| 9 | | G | V | L | L | S | R | K | | VGSGVLLSRK |
| | | | | | | | | | | (SEQ ID NO: 30) |
| | | | | | | | | | | SEQ ID NO: 17 |
| | | | | | | | | | | SEQ ID NO: 18 |
| | | | | | | | | | | SEQ ID NO: 19 |
| 10 | Peptide** | L | V | N | M | A | E | G | D | SEQ ID NO: 10 |

Positions Pₙ and Pₙ′ (wherein n is 1, 2, 3, 4, etc.) refer to the positioning of the amino acids in a peptide relative to the site of cleavage, indicated by the double vertical bar. Position numbers increase distally from the scissile bond.
*Synthetic peptide based upon the amino acid sequence of Notch protein (Accession number AAG33848). Memapsin 2 cleaves the Notch peptide at three sites.
**Synthetic peptide based upon the processing site of Presenilin-1 (Accession number P49768, Sherrington et al., Nature 375(6534): 754-760(1995), the teachings of which are hereby incorporated by reference in its entirety).

### EXAMPLE 2

### Activation of Pro-Memapsin 2 and Enzyme Kinetics

Incubation in 0.1 M sodium acetate, pH 4.0, for 16 h at 22° C. auto-catalytically converted pro-M2_{pd} to M2_{pd}. For initial hydrolysis tests, two synthetic peptides were separately incubated with pro-M2_{pd} in 0.1 M Na acetate, pH 4.0 for different periods ranging from 2 to 18 h. The incubated samples were subjected to LC/MS for the identification of the hydrolytic products. For kinetic studies, the identified HPLC (Beckman System Gold) product peaks were integrated for quantitation. The Kₘ and k_{cat} values for presenilin 1 and Swedish APP peptides (Table 1) were measured by steady-state kinetics. The individual Kₘ and k_{cat} values for APP peptide could not be measured accurately by standard methods, so its k_{cat}/Kₘ value was measured by competitive hydrolysis of mixed substrates against presenilin 1 peptide (Fersht, A. “Enzyme Structure and Mechanism”, 2^{nd} Ed., W.H. Freeman and Company, New York. (1985), the teachings of which are incorporated herein in their entirety).

The conversion of pro-M2_{pd} at pH 4.0 to smaller fragments was shown by SDS-polyacrylamide electrophoresis. The difference in migration between pro-M2_{pd} and converted enzyme is evident in a mixture of the two.

### EXAMPLE 3

### Design and Synthesis of Memapsin 2 Inhibitors OM99-1 and OM99-2

Based on the results of specificity studies of memapsin 2, it was predicted that good residues for positions P1 and P1′ would be Leu and Ala. It was subsequently determined from the specificity data that P1′ preferred small residues, such as Ala and Ser. However, the crystal structure (determined below) indicates that this site can accommodate a lot of larger residues. It was demonstrated that P1′ of memapsin 2 is the position with the most stringent specificity requirement where residues of small side chains, such as Ala, Ser, and Asp, are preferred. Ala was selected for P1′ mainly because its hydophobicity over Ser and Asp is favored for the penetration of the blood-brain barrier, a requirement for the design of a memapsin 2 inhibitor drug for treating Alzheimer's disease. Therefore, inhibitors were designed to place a transition-state analogue isostere between Leu and Ala (shown as Leu*Ala, where * represents the transition-state isostere, —CH(OH)—CH₂—) and the subsite P4, P3, P2, P2′, P3′ and P4′ are filled with the beta-secretase site sequence of the Swedish mutant from the beta-amyloid protein.

OM99-1: Val-Asn-Leu*Ala-Ala-Glu-Phe (SEQ ID NO: 20)

OM99-2: Glu-Val-Asn-Leu*Ala-Ala-Glu-Phe (SEQ ID NO: 21)

The Leu*Ala dipeptide isostere was synthesized as follows:

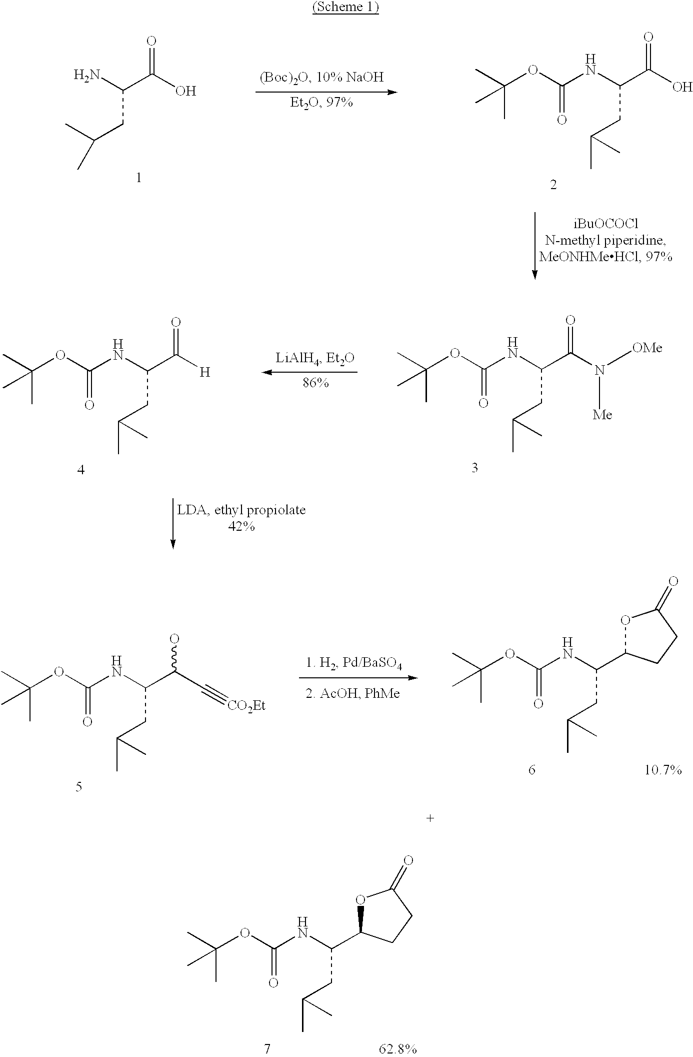

The Leu-Ala dipeptide isostere for the M₂-inhibitor was prepared from L-leucine. A bolded number in the following description, e.g. 2, 3, 4, etc, refers to the respective numbered compound in synthesis schemes 1, 2, and 3. The bolded number is also referred to herein as “compound”, e.g. compound 2, of the synthesis scheme.

As shown in Scheme 1, L-leucine was protected as its BOC-derivative 2 by treatment with BOC₂O in the presence of 10% NaOH in diethyl ether for 12 h. Boc-leucine 2 was then converted to Weinreb amide 3 by treatment with isobutyl chcloroformate and N-methylpiperidine followed by treatment of the resulting mixed anhydride with N,O-dimethylhydroxylamine (Nahm and Weinreb, Tetrahedron Letters 1981, 32: 3815, the teachings of which are incorporated herein in their entirety). Reduction of 3 with lithium aluminum hydride in diethyl ether provided the aldehyde 4. Reaction of the aldehyde 4 with lithium propiolate derived from the treatment of ethyl propiolate and lithium diisopropylamide afforded the acetylenic alcohol 5 as an inseparable mixture of diastereomers (5.8: 1) in 42% isolated yield (Fray, Kaye and Kleinman, J. Org. Chem. 1986, 51: 4828-33, the teachings of which are incorporated herein in their entirety). Catalytic hydrogenation of 5 over Pd/BaSO₄ followed by acid-catalyzed lactonization of the resulting gamma-hydroxy ester with a catalytic amount of acetic acid in toluene at reflux, furnished the gamma-lactone 6 and 7 in 73% yield.

The isomers were separated by silica gel chromatography by using 40% ethyl acetate in hexane as the eluent. Introduction of the methyl group at C-2 was accomplished by stereoselective alkylation of 7 with methyl iodide (Scheme 2). Thus, generation of the dianion of lactone 7 with lithium hexamethyldisilazide (2.2 equivalents) in tetrahydrofuran at −78° C. (30 min) and alkylation with methyl iodide (1.1 equivalents) for 30 min at −78° C., followed by quenching with propionic acid (5 equivalents), provided the desired alkylated lactone 8 (76% yield) along with a small amount (less than 5%) of the corresponding epimer (Ghosh and Fidanze, 1998 J. Org. Chem. 1998, 63, 6146-54, the teachings of which are incorporated herein in their entirety). The epimeric cis-lactone was removed by column chromatography over silica gel using a mixture (3:1) of ethyl acetate and hexane as the solvent system. The stereochemical assignment of alkylated lactone 8 was made based on extensive ¹H-NMR NOE experiments. Aqueous lithium hydroxide promoted hydrolysis of the lactone 8 followed by protection of the gamma-hydroxyl group with tert-butyldimethylsilyl chloride in the presence of imidazole and dimethylaminopyridine in

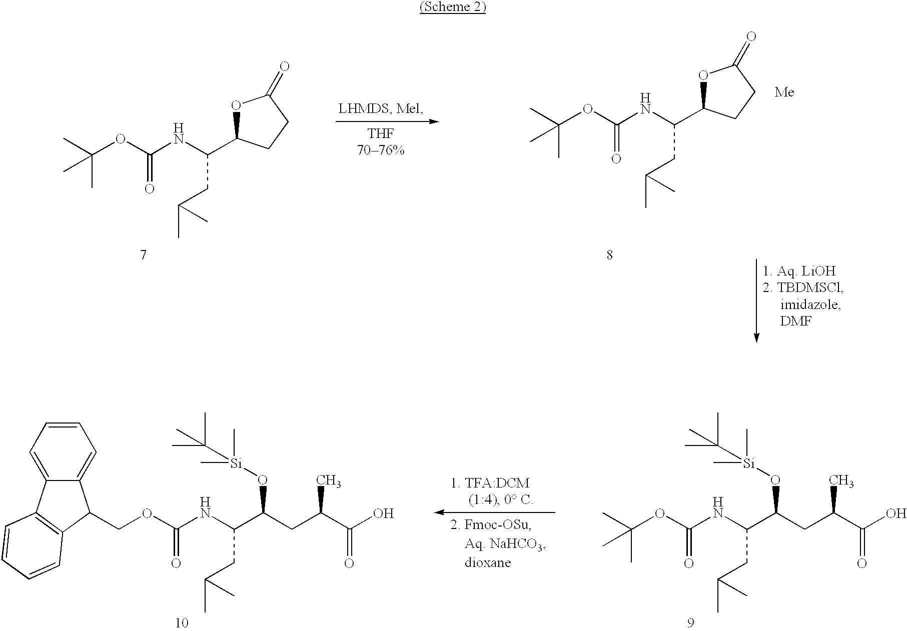

dimethylformamide afforded the acid 9 in 90% yield after standard work-up and chromatography. Selective removal of the BOC-group was effected by treatment with trifluoroacetic acid in dichloromethane at 0° C. for 1 h. The resulting amine salt was then reacted with commercial (Aldrich, Milwaukee) Fmoc-succinimide derivative in dioxane in the presence of aqueous NaHCO₃ to provide the Fmoc-protected L*A isostere 10 in 65% yield after chromatography. Protected isostere 10 was utilized in the preparation of a random sequence inhibitor library.

Experimental Procedure

N-(tert-Butoxycarbonyl)-L-Leucine (Compound 2).

To the suspension of 10 g (76.2 mmol ) of L-leucine in 140 mL of diethyl ether was added 80 mL of 10% NaOH. After all solid dissolves, 20 mL (87.1 mmol) of BOC₂O was added to the reaction mixture. The resulting reaction mixture was stirred at 23° C. for 12 h. After this period, the layers were separated and the aqueous layer was acidified to pH 1 by careful addition of 1 N aqueous HCl at 0° C. The resulting mixture was extracted with ethyl acetate (3×100 mL). The organic layers were combined and washed with brine and dried over anhydrous Na₂SO₄. The solvent was removed under reduced pressure to provide title product which was used directly for next reaction without further purification (yield, 97%). ¹H NMR (400 MHz, CDCl₃) δ 4.89 (broad d, 1H, J=8.3 Hz), 4.31 (m, 1H), 1.74-1.49 (m, 3H), 1.44 (s, 9H), 0.95 (d, 6H, J=6.5 Hz).

N-(tert-Butoxycarbonyl)-L-leucine-N′-methoxy-N′-methyla-mide (Compound 3).

To a stirred solution of N,O-dimethylhydroxyamine hydrochloride (5.52 g, 56.6 mmol) in dry dichloromethane (25 mL) under N₂ atmosphere at 0° C., -methylpiperidine (6.9 mL, 56.6 mmol) was added dropwise. The resulting mixture was stirred at 0° C. for 30 min. In a separate flask, N-(tert-butyloxycarbonyl)-L-leucine (2) (11.9 g, 51.4 mmol) was dissolved in a mixture of THF (45 mL) and dichloromethane (180 mL) under N₂ atmosphere. The resulting solution was cooled to −20° C. To this solution was added 1-methylpiperidine (6.9 mL, 56.6 mmol) followed by isobutyl chloroformate (7.3 mL, 56.6 mmol). The resulting mixture was stirred for 5 minutes at −20° C. and the above solution of N,O-dimethylhydroxyamine was added to it. The reaction mixure was kept −20° C. for 30 minutes and then warmed to 23° C. The reaction was quenched with water and the layers were separated. The aqueous layer was extracted with dichloromethane (3×100 mL). The combined organic layers were washed with 10% citric acid, saturated sodium bicarbonate, and brine. The organic layer was dried over anhydrous Na₂SO₄ and concentrated under the reduced pressure. The residue was purified by flash silica gel chromatography (25% ethyl acetate/hexane) to yield the title compound 3 (13.8 g, 97%) as a pale yellow oil. ¹H NMR (400 MHz, CDCl₃) δ 5.06 (broad d, 1H, J=9.1 Hz), 4.70 (m, 1H), 3.82 (s, 3H), 3.13 (s, 3H), 1.70 (m, 1H), 1.46-1.36 (m, 2H) 1.41 (s, 9H), 0.93 (dd, 6H, J=6.5, 14.2 Hz).

N-(tert-Butoxycarbonyl)-L-leucinal (Compound 4).

To a stirred suspension of lithium aluminum hydride (770 mg, 20.3 mmol) in dry diethyl ether (60 mL) at −40° C. under N₂ atmosphere, was added N-tert-butyloxycarbonyl-L-leucine-N′-methoxy-N′-methylamide (5.05 g, 18.4 mmol) in diethyl ether (20 mL). The resulting reaction mixture was stirred for 30 min. After this period, the reaction was quenched with 10% NaHSO₄ solution (30 mL). The resulting reaction mixture was then warmed to 23° C. and stirred at that temperature for 30 min. The resulting solution was filtered and the filter cake was washed by two portions of diethyl ether. The combined organic layers were washed with saturated sodium bicarbonate, brine and dried over anhydrous MgSO₄. Evaporation of the solvent under reduced pressure afforded the title aldehyde 4 (3.41 g) as a pale yellow oil. The resulting aldehyde was used immediately without further purification. ¹H NMR (400 MHz, CDCl₃) δ 9.5 (s, 1H), 4.9 (s, 1H), 4.2 (broad m, 1H), 1.8-1.6 (m, 2H), 1.44 (s, 9H), 1.49-1.39 (m, 1H), 0.96 (dd, 6H, J=2.7, 6.5 Hz).

Ethyl (4S,5S)- and (4R,5S)-5-[(tert-Butoxycarbonyl)amino]-4-hydroxy-7-methyloct-2-ynoate (Compound 5).

To a stirred solution of diisopropylamine (1.1 mL, 7.9 mmol) in dry THF (60 mL) at 0° C. under N₂ atmosphere, was added n-BuLi (1.6 M in hexane, 4.95 mL, 7.9 mmol) dropwise. The resulting solution was stirred at 0° C. for 5 min and then warmed to 23° C. and stirred for 15 min. The mixture was cooled to −78° C. and ethyl propiolate (801 mL) in THF (2 mL) was added dropwise over a period of 5 min. The mixture was stirred for 30 min, after which N-Boc-L-leucinal 4 (1.55 g, 7.2 mmol) in 8 mL of dry THF was added. The resulting mixture was stirred at −78° C. for 1 h. After this period, the reaction was quenched with acetic acid (5 mL) in THF (20 mL). The reaction mixure was warmed up to 23° C. and brine solution was added. The layers were separated and the organic layer was washed with saturated sodium bicarbonate and dried over Na₂SO₄. Evaporation of the solvent under reduced pressure provided a residue which was purified by flash silica gel chromatography (15% ethyl acetate/hexane) to afford a mixture (3:1) of acetylenic alcohols 5 (0.96 g, 42%). ¹H NMR (300 MHz, CDCl₃) δ 4.64 (d, 1H, J=9.0 Hz), 4.44 (broad s, 1H), 4.18 (m, 2H ), 3.76 (m, 1H), 1.63 (m, 1H), 1.43-1.31 (m, 2H), 1.39 (s, 9H), 1.29-1.18 (m, 3H), 0.89 (m, 6H).

(5S,1′S)-5-[1′-[(tert-Butoxycarbonyl)amino]-3′-methylbutyl]-dihydrofuran-2(3H)-one (Compound 7).

To a stirred solution of the above mixture of acetylenic alcohols (1.73 g, 5.5 mmol ) in ethyl acetate (20 mL) was added 5% Pd/BaSO₄ (1 g). The resulting mixture was hydrogenated at 50 psi for 1.5 h. After this period, the catalyst was filtered off through a plug of Celite and the filtrate was concentrated under reduced pressure. The residue was dissolved in toluene (20 mL) and acetic acid (100 μL). The reaction mixure was refluxed for 6 h. After this period, the reaction was cooled to 23° C. and the solvent was evaporated to give a residue which was purified by flash silica gel chromatography (40% diethyl ether/hexane) to yield the (5S,1S′)-gamma-lactone 7 (0.94 g, 62.8 and the (5R,1S′)-gamma-lactone 6 (0.16 g, 10.7%). Lactone 7: ¹H NMR (400 MHz, CDCl₃) δ 4.50-4.44 (m, 2H), 3.84-3.82 (m, 1H), 2.50 (t, 2H, J=7.8 Hz), 2.22-2.10 (m, 2H), 1.64-1.31 (m, 3H), 1.41 (s, 9H), 0.91 (dd, 6H, J=2.2, 6.7 Hz); ¹³C NMR (75 MHz, CDCl₃) 6177.2, 156.0, 82.5, 79.8, 51.0, 42.2, 28,6, 28.2, 24.7, 24.2, 23.0, 21.9.

(3R,5S,1′S)-5-[1′-[(tert-Butoxycarbonyl)amino)]-3′-methylbutyl]-3-methyl Dihydrofuran-2(3H)-one (Compound 8).

To a stirred solution of the lactone 7 (451.8 mg, 1.67 mmol) in dry THF (8 mL) at −78° C. under N₂ atmosphere, was added lithium hexamethyldisilazane (3.67 mL, 1.0 M in THF) over a period of 3 min. The resulting mixture was stirred at −78° C. for 30 min to generate the lithium enolate. After this period, MeI (228 μL) was added dropwise and the resulting mixture was stirred at −78° C. for 20 min. The reaction was quenched with saturated aqueous NH₄Cl solution and was allowed to warm to 23° C. The reaction mixture was concentrated under reduced pressure and the residue was extracted with ethyl acetate (3×100 mL). The combined organic layers were washed with brine and dried over anhydrous Na₂SO₄. Evaporation of the solvent afforded a residue which was purified by silica gel chromatography (15% ethyl acetate/hexane) to furnish the alkylated lactone 8 (0.36 g, 76%) as an amorphous solid. ¹H NMR (300 MHz, CDCl₃) δ 4.43 (broad t, 1H, J=6.3 Hz), 4.33 (d, 1H, J=9.6 Hz), 3.78 (m, 1H), 2.62 (m, 1H), 2.35 (m, 1H), 1.86 (m, 1H), 1.63-1.24 (m, 3H), 1.37 (s, 9H), 1.21 (d, 3H, J=7.5 Hz), 0.87 (dd, 6H, J=2.6, 6.7 Hz); ¹³C NMR (75 MHz, CDCl₃) δ 180.4, 156.0, 80.3, 79.8, 51.6, 41.9, 34.3, 32.5, 28.3, 24.7, 23.0, 21.8, 16.6.

(2R,4S,5S)-5-[(tert-Butoxycarbonyl)amino]-4-[(tert-butyldimethylsilyl)oxy]-2,7-dimethyloctanoic Acid (Compound 9)

To a stirred solution of lactone 8 (0.33 g, 1.17 mmol) in THF (2 mL) was added 1 N aqueous LiOH solution (5.8 mL). The resulting mixture was stirred at 23° C. for 10 h. After this period, the reaction mixture was concentrated under reduced pressure and the remaining aqueous residue was cooled to 0° C. and acidified with 25% citric acid solution to pH 4. The resulting acidic solution was extracted with ethyl acetate (3×50 mL). The combined organic layers were washed with brine, dried over Na₂SO₄ and concentrated to yield the corresponding hydroxy acid (330 mg) as a white foam. This hydroxy acid was used directly for the next reaction without further purification.

To the above hydroxy acid (330 mg, 1.1 mmol) in anhydrous DMF was added imidazole (1.59 g, 23.34 mmol) and tert-butyldimethylchlorosilane (1.76 g, 11.67 mmol). The resulting mixture was stirred at 23° C. for 24 h. After this period, MeOH (4 mL) was added and the mixture was stirred for 1 h. The mixure was diluted with 25% citric acid (20 mL) and was extracted with ethyl acetate (3×20 mL). The combined extracts were washed with water, brine and dried over anhydrous Na₂SO₄. Evaporation of the solvent gave a viscous oil which was purified by flash chromatography over silica gel (35% ethyl acetate/hexane) to afford the silyl protected acid 9 (0.44 g, 90%). IR (neat) 3300-3000 (broad), 2955, 2932, 2859, 1711 cm⁻¹; ¹H NMR (400 MHz, DMSO-d⁶, 343 K) delta 6.20 (broad s, 1H), 3.68 (m, 1H), 3.51 (broad s, 1H), 2.49-2.42 (m, 1H), 1.83 (t, 1H, J=10.1 Hz), 1.56 (m, 1H), 1.37 (s, 9H), 1.28-1.12 (m, 3H), 1.08 (d, 3H, J=7.1 Hz), 0.87 (d, 3H, J=6.1 Hz) 0.86 (s, 9H), 0.82 (d, 3H, J=6.5 Hz), 0.084 (s, 3H), 0.052 (s, 3H).

(2R,4S,5S)-5-[fluorenylmethyloxycarbonyl)amino]-4-[(tert-butyldi-methyl silyl)oxy]-2,7-dimethyloctanoic Acid (Compound 10).

To a stirred solution of the acid 9 (0.17 g, 0.41 mmol) in dichloromethane (2 mL) at 0° C. was added trifluoroacetic acid (500 μL). The resulting mixture was stirred at 0° C. for 1 h and an additional portion (500 μL) of trifluoroacetic acid was added to the reaction mixture. The mixture was stirred for an additional 30 min and the progress of the reaction was monitored by TLC. After this period, the solvents were carefully removed under reduced pressure at a bath temperature not exceeding 5° C. The residue was dissolved in dioxane (3 mL) and NaHCO₃ (300 mg) in 5 mL of H₂O. To this solution was added Fmoc-succinimide (166.5 mg, 0.49 mmol) in 5 mL of dioxane. The resulting mixture was stirred at 23° C. for 8 h. The mixure was then diluted with H₂O (5 mL) and acidified with 25% aqueous citric acid to pH 4. The acidic solution was extracted with ethyl acetate (3×50 mL). The combined extracts were washed with brine, dried over Na₂SO₄ and concentrated under reduced pressure to give a viscous oil residue. Purification of the residue by flash chromatography over silica gel afforded the Fmoc-protected acid 10 (137 mg, 61%) as a white foam. ¹H NMR (400 MHz, DMSO-d⁶, 343 K) δ 7.84 (d, 2H, J=7.4 Hz), 7.66 (d, 2H, J=8 Hz), 7.39 (t, 2H, J=7.4 Hz), 7.29 (m, 2H), 6.8 (s, 1H), 4.29-4.19 (m, 3H), 3.74-3.59 (m, 2H), 2.49 (m, 1H), 1.88 (m, 1H), 1.58 (m, 1H), 1.31-1.17 (m, 3H), 1.10 (d, 3H, J=7.1 Hz), 0.88 (s, 9H), 0.82 (d, 6H, J=6.2 Hz), 0.089 (s, 3H), 0.057 (s, 3H).

The synthesis of OM99-1 and OM99-2 were accomplished using solid state peptide synthesis procedure in which Leu*Ala was incorporated in the fourth step. The synthesized inhibitors were purified by reverse phase HPLC and their structure confirmed by mass spectrometry.

### EXAMPLE 4

### Design, Synthesis and Analysis of Additional Inhibitors.

Various substrate analogues other than OM99-1 and OM99-2 can be similarly designed and synthesized. For example, sixty six additional inhibitor analogues, MMI-001 to MMI-062, MMI-065, MMI-066, MMI-070 and MMI-071, all of which resemble an isostere of the active site of memapsin 2, were designed. The synthesis of the additional substrate analogues follows that for OM99-1 and OM99-2. The chemical structures of the additional substrate analogues are listed in Table 3. The general synthesis of various inhibitors are outlined in Scheme 3.

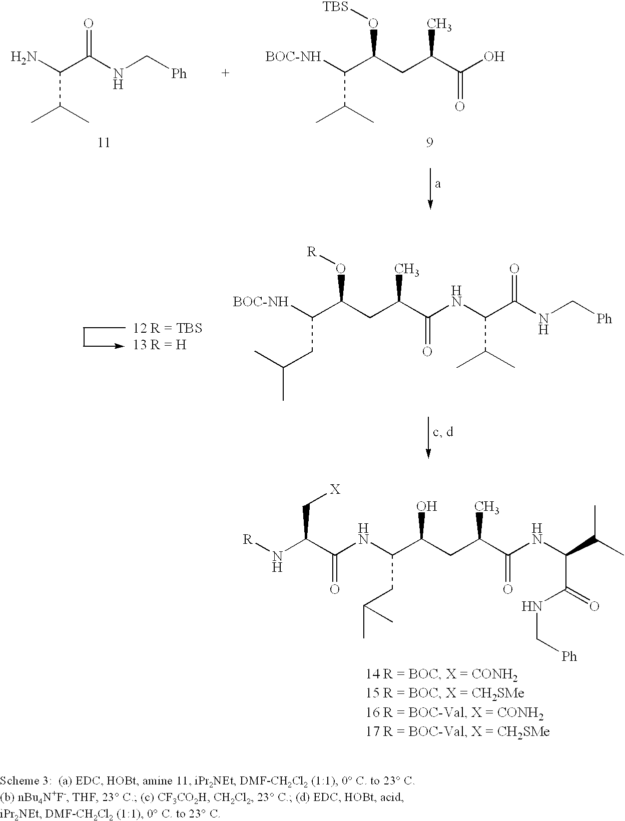

As scheme 3 demonstrates, using a standard peptide coupling procedure, valine derivative 11 was reacted with the dipeptide isostere 9 in the presence of N-ethyl-N′-(dimethylaminopropyl)carbodiimide hydrochloride, triethylamine and 1-hydroxybenzotriazole hydrate in a mixture of DMF and CH₂Cl₂ to afford the amide derivative 12. Removal of the silyl protecting group by treatment with tetrabutylammonium fluoride in THF afforded inhibitor 13 (MMI-001). Exposure of 13 to trifluoroacetic acid in CH₂Cl₂ resulted in the removal of BOC group. Coupling of the resulting amine with BOC-asparagine provided inhibitor 14 (MMI-01 1). Treatment of 14 with trifluoroacetic acid and coupling of the resulting amine with BOC-valine under standard conditions afforded inhibitor 16 (MMI-012). For the synthesis of inhibitor MMI-15, compound 13 was reacted with trifluoroacetic acid and the resulting amine was coupled with BOC-methionine to afford the inhibitor 15 (MMI-015). Removal of the BOC group of 16 and coupling of the resulting amine with BOC-valine provided the inhibitor 17 (MMI-017).

Preparation of inhibitors MMI-001, MMI-011, MMI-012, MMI-015 and MMI-17:

Preparation of Valine Derivative (Compound 11):

N-Boc Valine (500 mg. 2.30 mmol) and benzylamine (0.50 mL, 4.60 mmol) were dissolved in CH₂Cl₂ (20 mL) and DMF (2 mL). To this solution, HOBt (373 mg, 2.76 mmol) and EDC (529 mg, 2.76 mmol), and diisopropylethylamine (2.4 mL, 13.80 mmol) were added successively at 0° C. After the addition, the reaction mixture was allowed to warm up to 23° C. and stirred overnight. The mixture was poured into sat. NaHCO₃ (aq). The resultant mixture was extracted with 30% EtOAc/hexane. The organic layer was washed with brine and dried over Na₂SO₄. Evaporation of the solvent under the reduced pressure gave a residue which was purified by flash column chromatography (30% EtOAc/hexane) to give 442 mg (63%) of coupling product. The resulting amine was dissolved in CH₂Cl₂ (20 mL). TFA (4 mL) was then added at room temperature. The reaction mixture was stirred for 0.5 hr. and it was concentrated under the reduced pressure. The amine 11 was obtained quantitative yield. ¹H NMR (500 MHz, CDCl₃) 0.87 (3H, d, J=6.9 Hz), 1.02 (3H, d, J=6.9 Hz), 2.00 (2H, br s), 2.37 (1H, m), 3.36 (1H, br s), 4.43-4.52 (2H, m), 7.27-7.37 (5H, m), 7.70 (1H, br s).

Preparation of Amide Derivative (Compound 12):

Dipeptide isostere 9 (41 mg. 0.10 mmol) and amine 11 (41 mg. 0.20 mmol) were dissolved in DMF (2.0 mL). To this solution, HOBt (20 mg, 0.15 mmol) and EDC (29 mg, 0.15 mmol), and diisopropylethylamine (0.2 mL) were added successively at 0° C. After the addition, the reaction mixture was allowed to warm up to 23° C. and was stirred overnight. The mixture was poured into sat. NaHCO₃ (aq). The mixture was extracted with 30% EtOAc/hexane. The organic layer was washed with brine and dried over Na₂SO₄. Evaporation of the solvent under the reduced pressure gave a residue which was purified by column chromatography (20% EtOAc/hexane) to give 55 md (95%) of amide 12. ¹H NMR (500 MHz, CDCl₃) 0.09 (3H, s), 0.10 (3H, s), 0.91 (9H, s), 0.92-0.98 (12H, m), 1.10 (3H, d, J=6.7 Hz), 1.25 (1H, m), 1.44 (1H, m), 1.46 (9H, s), 1.63 (1H, m), 1.74 (1H, br s), 1.80 (1H, m), 2.18 (1H, m), 2.56 (1H, m), 3.62-3.78 (2H, m), 4.13 (1H, m), 4.48-4.56 (3H, m), 6.35 (1H, br d, J=8.5 Hz), 6.41 (1H, br s), 7.26-7.40 (5H, m).

Preparation of Inhibitor MMI-001 (Compound 13):

To a solution of amide 12 (61 mg. 0.10 mmol) in THF (1.0 mL0 was added TBAF (1.0 M in THF:0.3 mL, 0.30 mmol) at 23° C. and it was stirred overnight. The reaction mixture was concentrated under reduced pressure. The residue was purified by column chromatography (40% EtOAc/hexane) to give MMI-001 (13, 41 mg, 83%). ¹H NMR (300 MHz, CDCl₃) 0.88-0.98 (12H, m), 1.15 (3H, d, J=6.9 Hz), 1.40-1.80 (5H, m), 1.43 (9H, s), 2.10 (1H, m), 2.60 (1H, m), 3.40-3.60 (2H, m), 4.00 (1H, m), 4.20-4.45 (3H, m), 4.70 (1H, m), 708-718 (5H, m).

Preparation of Inhibitor MMI-011 (Compound 14):

To a solution of 13 (44 mg, 0.089 mmol) in CH₂Cl₂ (1 mL) was added TFA (0.2 ml) at 23° C. After 0.5 hr, the reaction mixture was concentrated under reduced pressure. The residue was dissolved in DMF (4 mL). To this solution, N-Boc-asparagine (41 mg, 0.18 mmol), HOBt (24 mg, 0.18 mmol) and EDC (34 mg 0.18 mmol), and diisopropylethylamine (0.2 mL) were added successively at 0° C. After the addition, the raction mixture was allowed to warn up to 23° C. and was stirred overnight. The mixture was poured into sat. NaHCO₃ (aq). The mixture was extracted with EtOAc. The organic layer was washed with brine and dried over Na₂SO₄. Evaporation of the solvent under the reduced pressure gave a residue which was purified by column chromatography (4% MeOH/EtOAc) to give 12.5 mg (23%) of MMI-011 (14). ¹H NMR (500 MHz, CD₃OD) 0.85-1.00 (12H, m), 1.10 (3H, d, J=6.7 Hz), 1.20-1.50 (5H, m), 1.45 (9H, s), 1.60 (1H, m), 1.75 (1H, m), 2.05 (1H, m), 2.50-2.70 (3H, m), 3.45 (1H, m), 3.75 (1H, m), 4.10 (1H, m), 4.30-4.40 (3H, m), 7.20-7.30 (5H, m).

Preparation of Inhibitor MMI-015 (Compound 15):

To a solution of compound 13 (64 mg. 0.13 mmol) in CH₂Cl₂ (4 mL) was added TFA (1 ml) at 23° C. After 0.5 hr, the reaction mixture was concentrated under reduced pressure. The residue was dissolved in DMF (4 mL). To this solution, N-Boc-methionine (65 mg, 0.26 mmol), HOBt (35 mg. 0.26 mmol) and EDC (50 mg, 0.26 mmol), and diisopropylethylamine (0.4 mL) were added successively at 0° C. After the addition, the reaction mixture was allowed to warm up to 23° C. and was stirred overnight. The mixture was poured into sat. NaHCO3 (aq). The mixture was extracted with EtOAc. The organic layer was washed with brine and dried over Na₂SO₄. Evaporation of the solvent under the reduced pressure gave a residue which was purified by column chromatography (80% EtOAc/liexane) to give 37.2 mg (46%) of MMI-015 (15). ¹H NMR (300 MHz, CD3OD) 0.80-1.00 (12H, m), 1.50 (3H, d, J=6.7 Hz), 1.20-2.10 (10H, m), 1.45 (9H, s), 2.10 (3H, s), 2.45-2.60 (2H, m), 2.70 (1H, m), 3.50 (1H, m), 3.90 (1H, m), 4.10-4.15 (2H, m), 4.30-4.42 (2H, m), 7.20-7.35 (5H, m).

Preparation of Inhibitor MMI-012 (Compound 16):

To a solution of 13 (8.4 mg, 0.014 mmol) in CH₂Cl₂ (1 mL) was added TFA (0.2 ml) at 23° C. After 0.5 hr, the reaction mixture was concentrated under reduced pressure. The residue was dissolved in DMF (1 mL). To this solution, N-Boc-valine (6 mg, 0.028 mmol), HOBt (3.8 mg, 0.028 mmol) and EDC (5.3 mg. 0.028 mmol), and diisopropylethylamine (0.2 mL) were added successively at 0° C. After the addition, the reaction mixture was allowed to warm up to 23° C. and was stirred overnight. The mixture was poured into sat. NaHCO₃ (aq). The mixture was extracted with EtOAc. The organic layer was washed with brine and dried over Na₂SO₄. Evaporation of the solvent under reduced pressure gave a residue which was purified by column chromatography (4% MeOH/EtOAc) to give 2.1 mg (21%) of inhibitor MMI-012 (16). ¹H NMR (400 MHz, CD₃OD) 0.80-1.00 (18H, m), 1.05 (3H, d, J=6.7 Hz), 1.10-1.50 (6H, m), 1.40 (9H, s), 1.55 (1H, m), 1.70 (1H, m), 2.00 (1H, m), 2.45-2.70 (3H, m), 3.45 (1H, m), 3.80-4.45 (6H, m), 7.15-7.30 (5H, m).

Preparation of Inhibitor MMI-107 (Compound 17):

To a solution of 15 (14.5 mg, 0.023 mmol) in CH₂Cl₂ (2 mL) was added TFA (0.5 ml) at 23° C. After 0.5 hr. the reaction mixture was concentrated under reduced pressure. The residue was dissolved in DMF (2 mL). To this solution, N-Boc-valine (10 mg., 0.046 mmol), HOBt (6.2 mg, 0.046 mmol) and EDC (8.8 mg, 0.046 mmol), and diisopropylethylamine (0.4 mL) were added successively at 0° C. After the addition, the reaction mixture was allowed to warm up to 23° C. and was stirred overnight. The mixture was poured into sat. NaHCO₃ (aq). The mixture was extracted with EtOAc. The organic layer was washed with brine and dried over Na₂SO₄. Evaporation of the solvent under the reduced pressure gave a residue which was purified by column chromatography (EtOAc) to give 5.5 mg (33%) of MMI-017 (17). Rf=(10% EtOAc/hexane); ¹H NMR (300 MHz, CD₃OD) 0.80-1.00 (18H, m), 1.10 (3H, d, J=6.7 Hz), 1.20-2.10 (11H, m), 1.43 (9H, s), 2.10 (3H, s), 2.40-2.60 (2H, m), 2.70 (1H, m), 3.50 (1H, m), 3.80-3.95 (2H, m), 4.18 (1H, m), 4.30-4.50 (3H, m), 7.20-7.30 (5H, m).

Enzyme activity of the isosteres was measured as described above, but with the addition of either OM99-1, OM99-2 or one of MMI-001-MMI-062, MMI-065, MMI-066, MMI-070 and MMI-071. OM99-1 inhibited recombinant memapsin with a Kᵢ calculated as 3×10⁻⁸ M. The substrate used was a synthetic fluorogenic peptide substrate. The inhibition of OM99-2 on recombinant memapsin 2 was measured using the same fluorogenic substrate. The Kᵢ value was determined to be 9.58×10⁻⁹ M.

The residues in P1 and P1′ are very important since the M2 inhibitor must penetrate the blood-brain barrier (BBB). The choice of Ala in P1′ facilitates the penetration of BBB. Analogues of Ala side chains will also work. For example, in addition to the methyl side chain of Ala, substituted methyl groups and groups about the same size like methyl or ethyl groups can be substituted for the Ala side chain. Leu at P1 can also be substituted by groups of similar sizes or with substitutions on Leu side chain. For penetrating the BBB, it is desirable to make the inhibitors smaller. One can therefore use OM99-1 as a starting point and discard the outside subsites P4, P3, P3′ and P4′. The retained structure Asn-Leu*Ala-Ala is then further evolved with substitutions for a tight-binding M2 inhibitor which can also penetrate the BBB.

The other substrate analogues, MMI-001 to MMI-071, were also tested for enzyme inhibition. For example, the Kᵢ values for MMI-017, MMI-070 and MMI-071 are comparable to that for OM99-2, indicating that they are also excellent memapsin inhibitors. The Kᵢ value for MMI-012, MMI-018, MMI-026, or MMI-066 is approximately one magnitude higher than that for OM99-2, indicating that they are competent candidates for memapsin inhibition. The Kᵢ value for each of the additional substrate analogues and its corresponding chemical structure are listed in Table 3.

TABLE 3
Chemical Structures and Kᵢ Values of Additional Substrate Analogues

| | Comp No./ |
|---|---|
| Structure | Kᵢ (nM) |
| 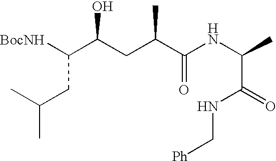 | MMI-001/ 3,738,100.0 |
| 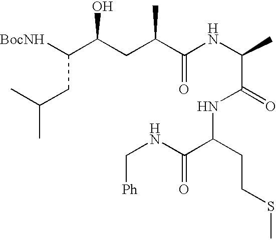 | MMI-002/ 430,940.0 |
| 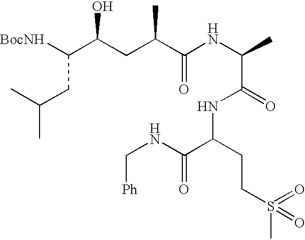 | MMI-003/ 2,617,000.0 |
| 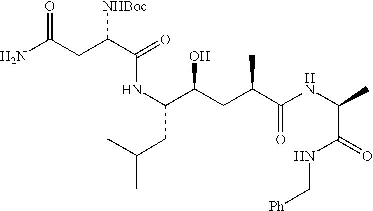 | MMI-004/ 22,423.0 |
| 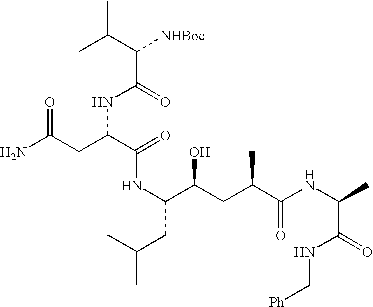 | MMI-005/ 61.4 |
| 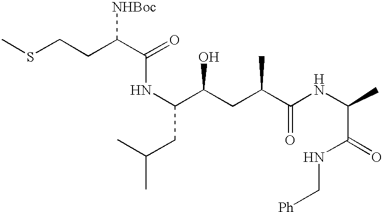 | MMI-006/ 63,288.0 |
| 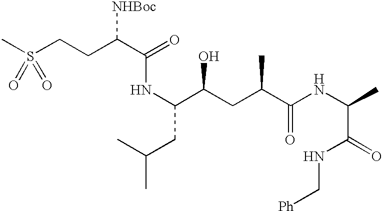 | MMI-007/ 49,877.0 |
| 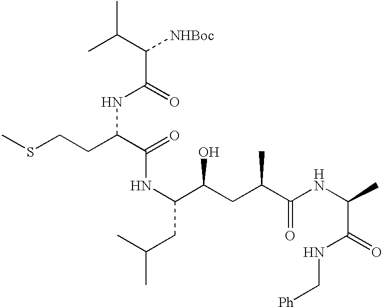 | MMI-008/ 73.6 |
| 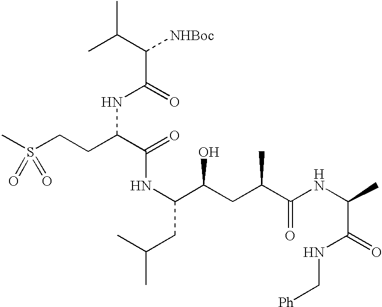 | MMI-009/ 89.9 |
| 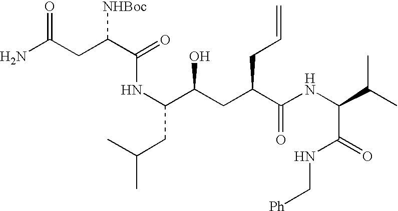 | MMI-010/ 29,403.0 |
| 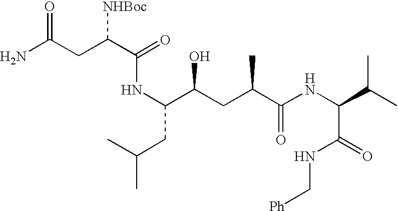 | MMI-011/ 3,134.0 |
| 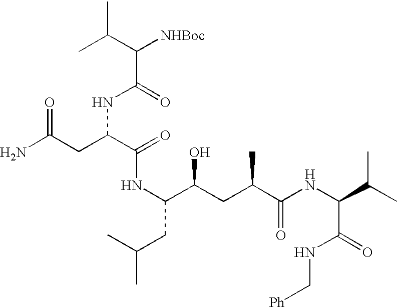 | MMI-012/ 5.9 |
| 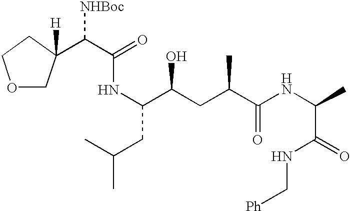 | MMI-013/ 184,160.0 |
| 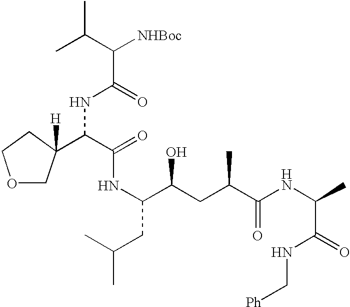 | MMI-014/ 2,777.8 |
| 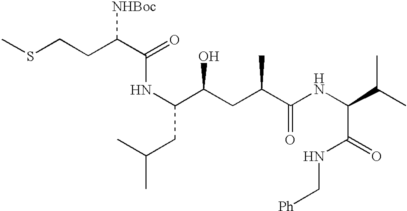 | MMI-015/ 5,808.0 |
| 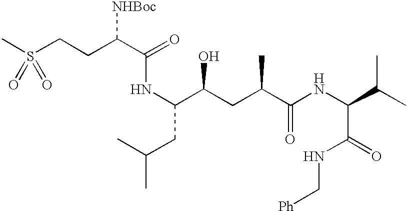 | MMI-016/ 5,640.0 |
| 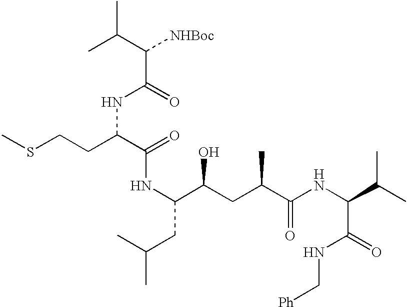 | MMI-017/ 2.5 |
| 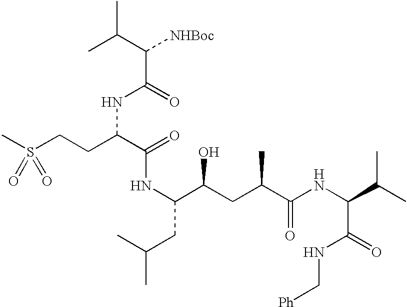 | MMI-018/ 8.0 |
| 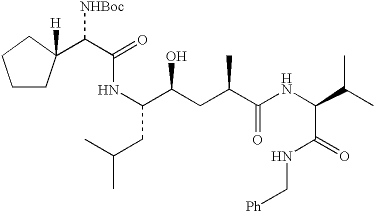 | MMI-019/ 14,500.0 |
| 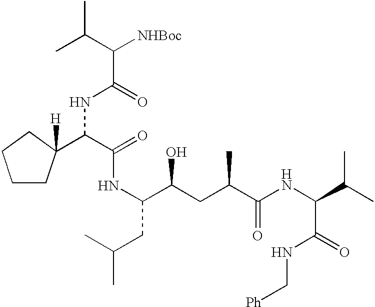 | MMI-020/ 1,605.2 |
| 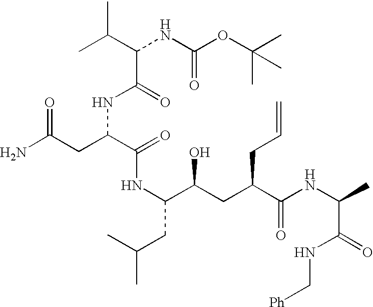 | MMI-021/ 112.2 |
| 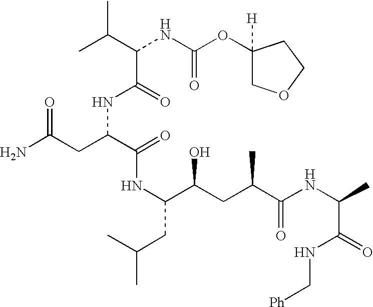 | MMI-022/ 11,766.0 |
| 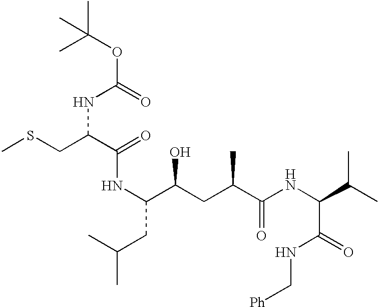 | MMI-023/ 15,273.0 |
| 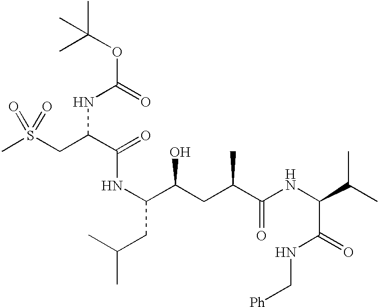 | MMI-024/ 1129.0 |
| 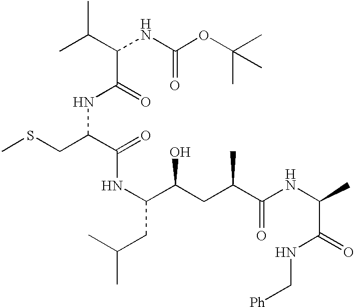 | MMI-025/ 50.1 |
| 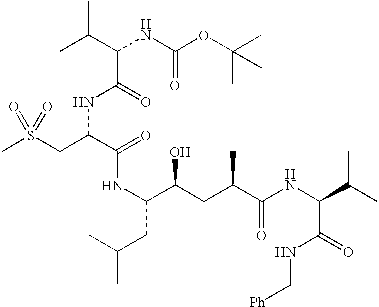 | MMI-026/ 9.4 |
| 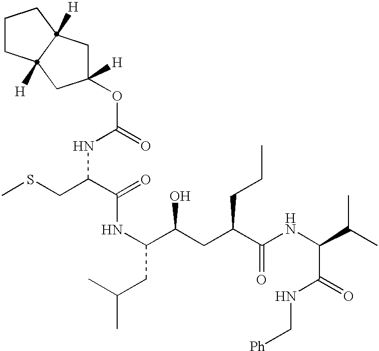 | MMI-027/ 11,547.3 |
| 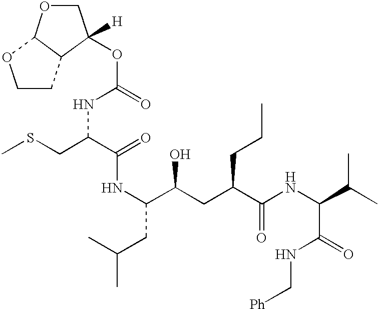 | MMI-028/ 9,867.0 |
| 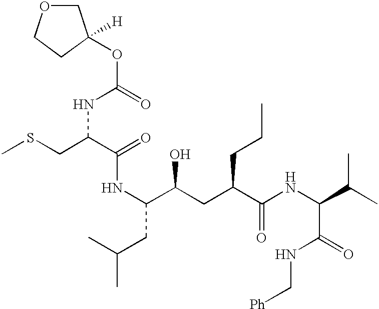 | MMI-029/ 1,696.6 |
| 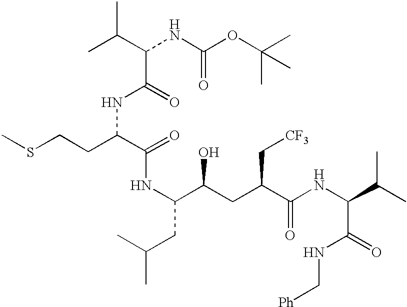 | MMI-030/ 8,517.0 |
| 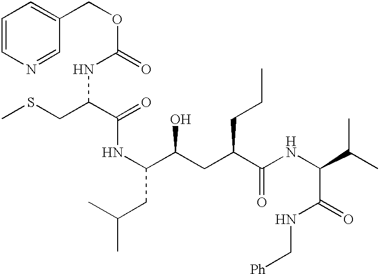 | MMI-031/ 863.7 |
| 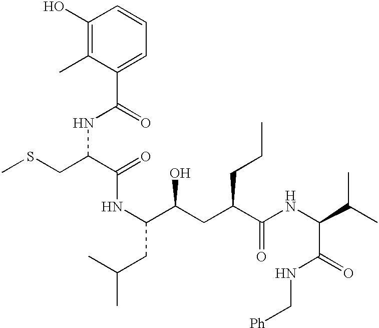 | MMI-032/ 1,341.0 |
| 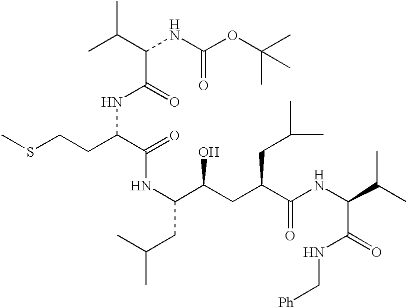 | MMI-033/ 16,300.0 |
| 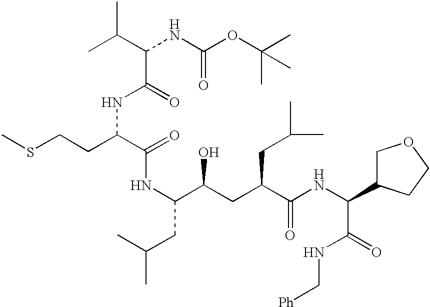 | MMI-034/ 228.8 |
| 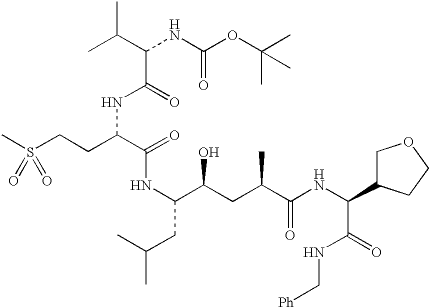 | MMI-035/ 426.3 |
| 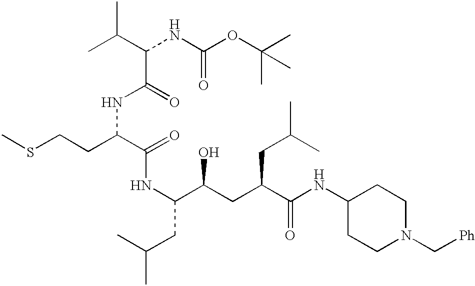 | MMI-036/ no inhibition |
| 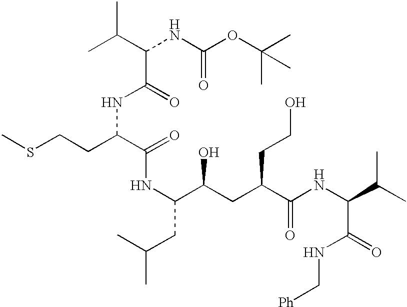 | MMI-037/ 3.9 |
| 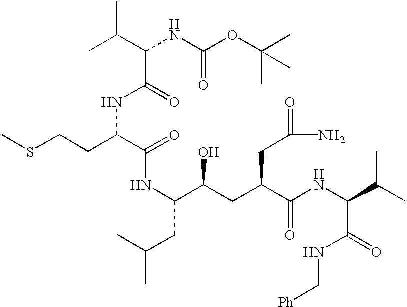 | MMI-038/ 106.0 |
| 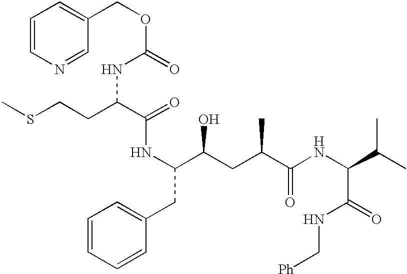 | MMI-039/ 1.4 |
| 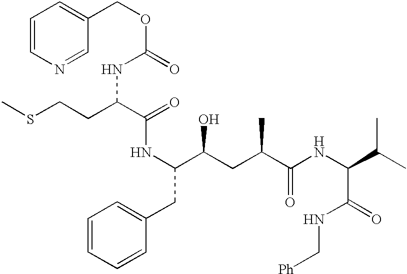 | MMI-040/ 2.1 |
| 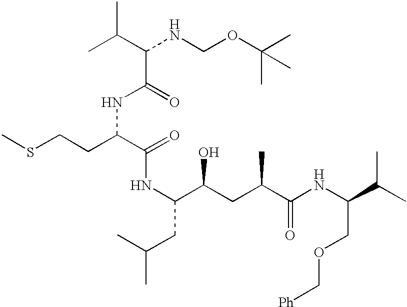 | MMI-041/ no inhibition |
| 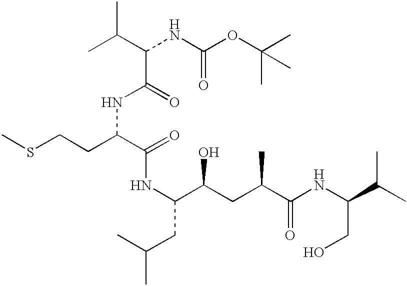 | MMI-042/ 36,484.1 |
| 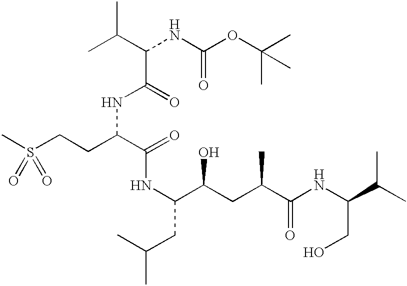 | MMI-043/ 49,188.9 |
| 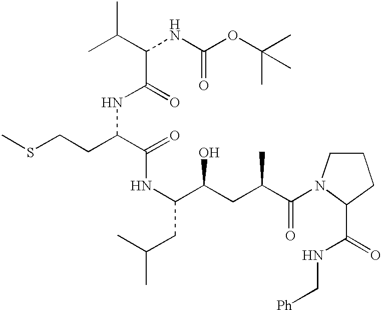 | MMI-044/ no inhibition |
| 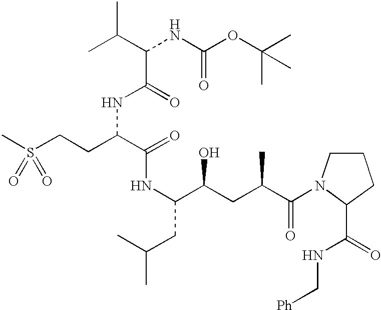 | MMI-045/ no inhibition |
| 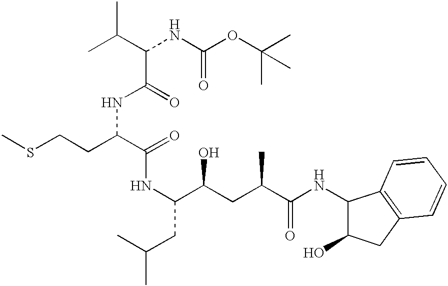 | MMI-046/ 149,900,000.0 |
| 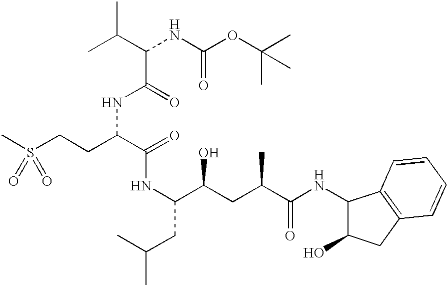 | MMI-047/ 110,600,000.0 |
| 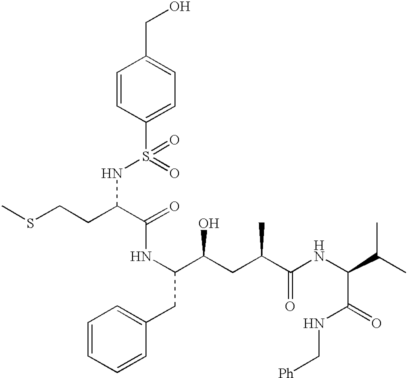 | MMI-048/ 54,8000,000.0 |
| 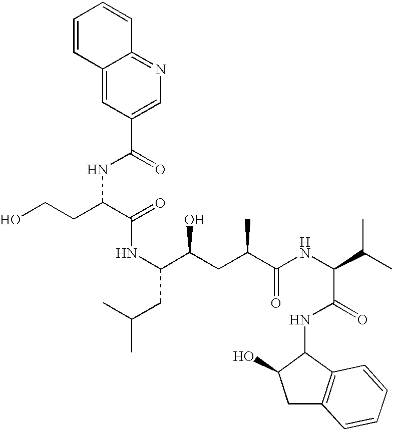 | MMI-049/ 198,000.0 |
| 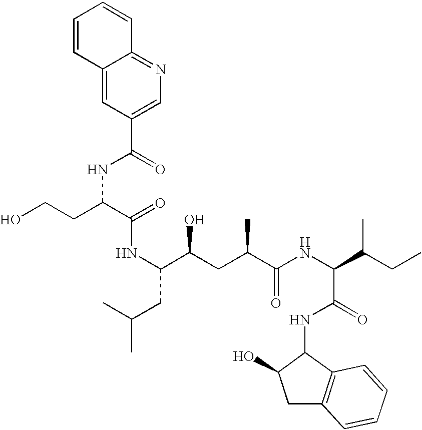 | MMI-050/ 146,000.0 |
| 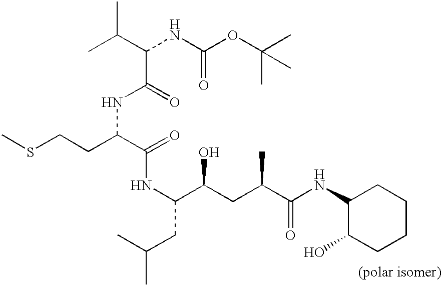 | MMI-051/ no inhibition |
| 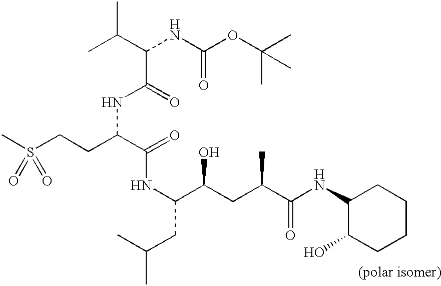 | MMI-052/ 5,790.0 |
| 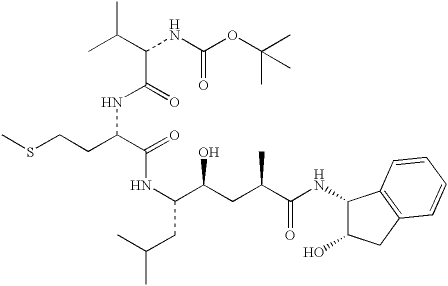 | MMI-053/ no inhibition |
| 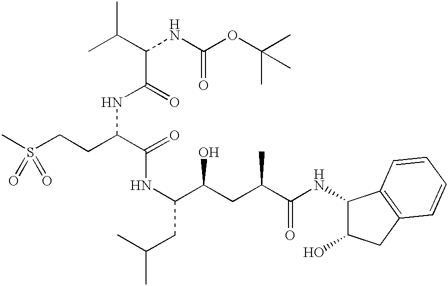 | MMI-054/ 469,000.0 |
| 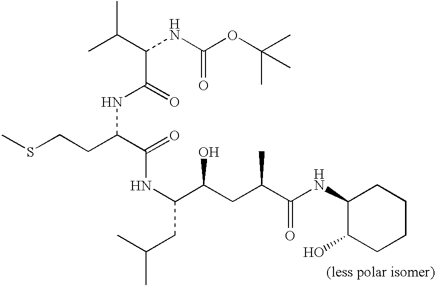 | MMI-055/ 631,000.0 |
| 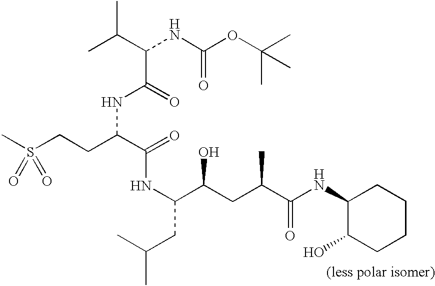 | MMI-056/ 205,000.0 |
| 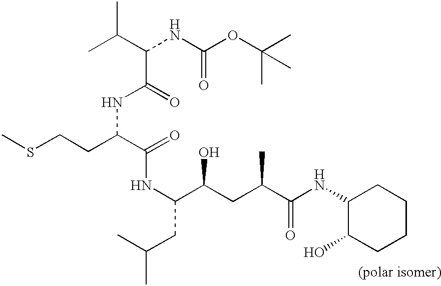 | MMI-057/ 73,300.0 |
| 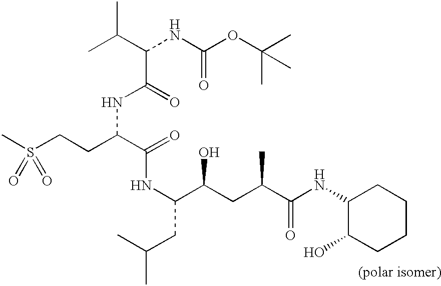 | MMI-058/ 408,000.0 |
| 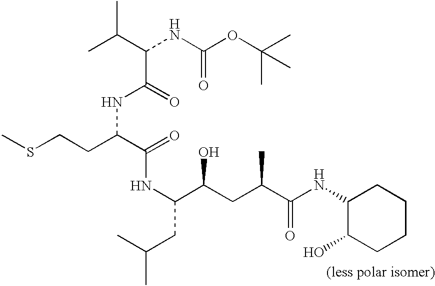 | MMI-059/ 66,300.0 |
| 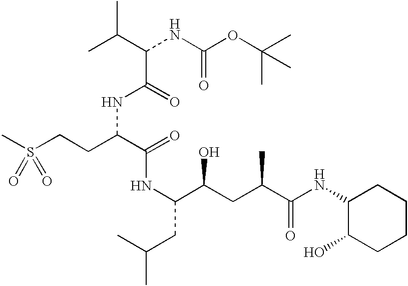 | MMI-060/ 100,000.0 |
| 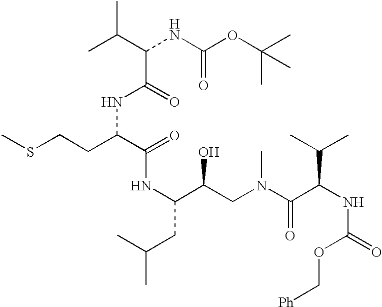 | MMI-061/ 15,460,000.0 |
| 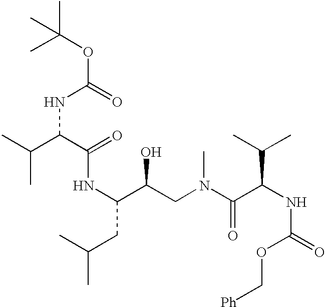 | MMI-062/ no inhibition |
| 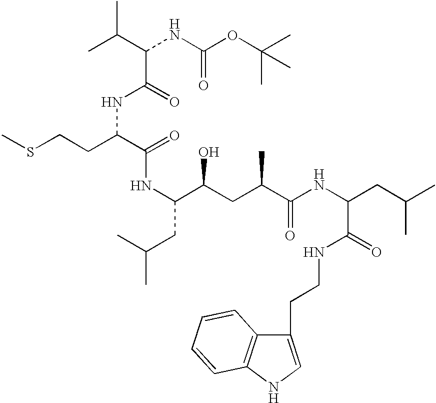 | MMI-065/ 40.0(97.4) |
| 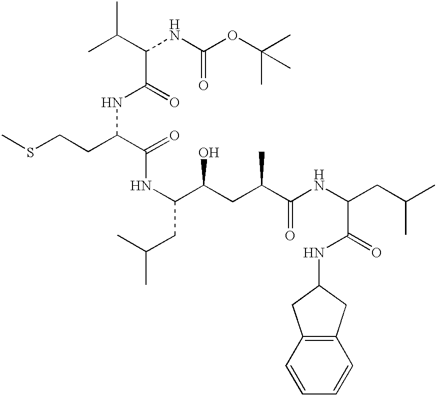 | MMI-066/ 21.2 |
| 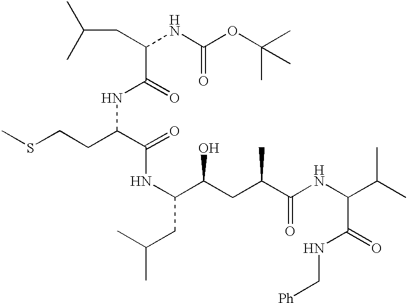 | MMI-070/ 2.0(4.9) |
| 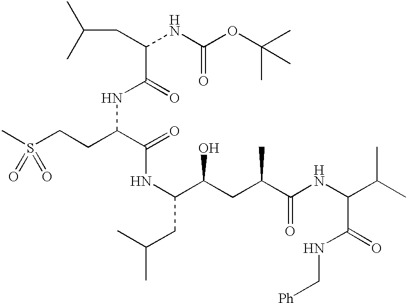 | MMI-071/ 4.0(1.9) |

### EXAMPLE 5

### Sub-site Specificity of Memapsin 2 (β-secretase)

The results obtained from OM99-2 demonstrate that by evoking all eight sub-sites with OM99-2, a high inhibition potency (Kᵢ=1.6 nM) can be achieved. However, clinically useful memapsin 2 inhibitors must be small (i.e., typically less than 500-700 daltons) in order to penetrate the blood-brain barrier to reach the brain. Detailed information on sub-site specificity of memapsin 2 would provide a better understanding for the design of small yet tight-binding inhibitors. The complete sub-site preference of memapsin 2 has been determined based on results from both substrate kinetics and the probing of random sequence inhibitor library.

Experimental Procedures

The Design of the Defined Substrate Mixtures

Peptide mixtures were synthesized to probe each of 8 positions in the template sequence EVNLAAEF (SEQ ID NO: 22), derived from the β-secretase cleavage site in APP and memapsin 2 inhibitor OM99-2 described above. For characterization of each position, an equimolar mixture of 7 amino acid derivatives were added in the appropriate cycle of solid phase synthesis (Research Genetics, Invitrogen, Huntsville, Ala.) resulting in a mixture of 7 peptides, differing by 1 of 7 amino acids at a single position. To facilitate the rapid quantitation of substrates and hydrolytic products, high throughput MALDI-TOF MS was used. Therefore, it was not possible to subsitute allcommon amino acid side chains (19 total, excluding cysteine) in a single mixture due to the identical masses of some amino acids which would prevent their identification. Therefore, the different amino acid substitutions were divided into three groups (Tables 4 and 5) to maximize their differences in mass and the full set of 19 varied amino acids (less cysteine) at each subsite were contained in 3 substrate mixtures in which the peptides were synthesized to incorporate each amino acid in a single position. A standard peptide was included in each group for the purpose of relating the quantitative information between mixtures, for calculation of relative k_{cat}/Kₘ. The substrate of known k_{cat}/Kₘ value serves as an internal standard for normalization of relative initial rates and the calculation of k_{cat}/Kₘ value of other substrates. For positions P₁′, P₂′, P₃′, and P₄′, the template sequence was extended by 4 residues at the C-terminus (template EVNLAAEFWHDR, SEQ ID NO: 23, Table 4) to facilitate their detection in MALDI-TOF MS. Four additional residues were likewise added on the N-terminus in the template to characterize specificity for positions P₁, P₂, P₃ and P₄ (template RWHHEVNLAAEF, SEQ ID NO: 24, Table 5).

TABLE 4
Peptide Substrate Mixture for Specificity
Determination of Memapsin 2 to the P₁′ Position

| | | | SEQ ID |
|---|---|---|---|
| | Amino Acid | Peptide Sequence | NO. |
| Substrate | A (alanine) | EVNLAAEFWHDR | 25 |
| mixture 1 | D (aspartic acid) | EVNLDAEFWHDR | 26 |
| | S (serine) | EVNLSAEFWHDR | 27 |
| | T (threonine) | EVNLTAEFWHDR | 28 |
| | I (isoleucine) | EVNLIAEFWHDR | 29 |
| | E (glutamic acid) | EVNLEAEFWHDR | 30 |
| | F (phenylalanine) | EVNLFAEFWHDR | 31 |
| Substrate | A (alanine) | EVNLAAEEWHDR | 32 |
| mixture 2 | G (glycine) | EVNLGAEFWHDR | 33 |
| | V (valine) | EVNLVAEFWHDR | 34 |
| | L (leucine) | EVNLLAEFWHDR | 35 |
| | K (lysine) | EVNLKAEFWHDR | 36 |
| | R (arginine) | EVNLRAEFWHDR | 37 |
| | W (tryptophan) | EVNLWAEFWHDR | 38 |
| Substrate | A (alanine) | EVNLAAEFWHDR | 39 |
| mixture 3 | P (proline) | EVNLPAEFWHDR | 40 |
| | N (asparagine) | EVNLNAEFWHDR | 41 |
| | Q (glutamine) | EVNLQAEFWHDR | 42 |
| | M (methionine) | EVNLMAEFWHDR | 43 |
| | H (histidine) | EVNLHAEFWHDR | 44 |
| | Y (tyrosine) | EVNLYAEFWHDR | 45 |

TABLE 5
Peptide Substrate Mixture for Specificity
Determination of Memapsin 2 to the P₁ Position

| | | | SEQ ID |
|---|---|---|---|
| | Amino Acid | Peptide Sequence | NO. |
| Substrate | F (phenylalanine) | RWHHEVNFAAEF | 46 |
| mixture 1 | D (aspartic acid) | RWHHEVNDAAEF | 47 |
| | S (serine) | RWHHEVNSAAEF | 48 |
| | T (threonine) | RWHHEVNTAAEF | 49 |
| | I (isoleucine) | RWHHEVNIAAEF | 50 |
| | E (glutamic acid) | RWHHEVNEAAEF | 51 |
| | G (glycine) | RWHHEVNGAAEF | 52 |
| Substrate | F (phenylalanine) | RWHHEVNFAAEF | 53 |
| mixture 2 | A (alanine) | RWHHEVNAAAEF | 54 |
| | V (valine) | RWHHEVNVAAEF | 55 |
| | L (leucine) | RWHHEVNLAAEF | 56 |
| | Q (glutamine) | RWHHEVNQAAEF | 57 |
| | M (methionine) | RWHHEVNMAAEF | 58 |
| | Y (tyrosine) | RWHHEVNYAAEF | 59 |
| Substrate | F (phenylalanine) | RWHHEVNFAAEF | 60 |
| mixture 3 | P (proline) | RWHHEVNPAAEF | 61 |
| | N (asparagine) | RWHHEVNNAAEF | 62 |
| | K (lysine) | RWHHEVNKAAEF | 63 |
| | R (arginine) | RWHHEVNRAAEF | 64 |
| | H (histidine) | RWHHEVNHAAEF | 65 |
| | W (tryptophan) | RWHHEVNWAAEF | 66 |

Initial Rate Determination by MALDI-TOF MS

Substrate mixtures were dissolved at 2 mg/ml in 10% glacial acetic acid, and diluted into appropriate concentration of NaOH to obtain a mixture of substrates in the μM range in sodium acetate at pH 4.1. Aliquots were equilibrated at 25 ^{K}C, and reactions were initiated by the addition of aliquots of memapsin 2. Aliquots (10 μl) were removed at time intervals and combined with MALDI-TOF matrix (α-hydroxycinnamic acid in acetone, 20 mg/ml) and immediately spotted in duplicate onto a stainless-steel MALDI sample plate. Samples were subjected to analysis by using MALDI-TOF mass spectrometry device, operated at 20,000 accelerating volts in positive mode with a 150 nsec delay, using a PE Biosystems Voyager DE instrument at the Molecular Biology Resource Center on campus. Ions with a mass-to-charge ratio (m/z) were detected in the range of 400-2000 amu (atomic mass units). Data were analyzed by the Voyager Data Explorer module to obtain ion intensity data for mass species of interest.

Random Sequence Inhibitor Library

The random sequence inhibitor library was based on the sequence of OM99-2 with random amino acids (less cysteine) at 4 subsite positions P₂, P₃, P₂′ and P₃′. Diisostere Leu*Ala (* represents hydroxyethylene transition-state isostere) was used in the synthesis to fix the positions P₁ and P₁′. Peptides were synthesized by solid-state peptide synthesis method and left attached on the resin beads. By using the ‘split-synthesis’ procedure (Lam et al., 1991), each of the resin beads contained only one -sequence while the sequence differed from bead to bead. The overall library sequence was:

(SEQ ID NO:67)

Gly-Xx1-Xx2-Leu*Ala-Xx3-Xx4-Phe-Arg-Met-Gly-Gly-
[Resin bead]
P₄ P₃ P₂ P₁ P₁′ P₂′ P₃′ P₄′

where Xxn residues (where n represents either 1, 2, 3, or 4) are randomized at each position with 19 amino acids. A shorter version of the peptides, starting at P₂′ (sequence: Xx3-Xx4-Phe-Arg-Met-Gly-Gly-[Resin bead] SEQ ID NO: 68), was also present in each bead with a ratio to the longer sequence at about 7:3. Without isostere, the short sequence would not bind memapsin 2 with significant strength but its presence was convenient for identifying the residues at by automated Edman degradation. The residues are identified from the randomized positions as follows:

| | Edman cycle # | | | |
|---|---|---|---|---|
| | 1 | 2 | 3 | 4 |
| Sequence 1, from the long sequence: | Gly | Xx1 | Xx2 | Leu |
| Sequence 2, from the short sequence: | Xx3 | Xx4 | Phe | Arg |

The assignment of Xx3 and Xx2 had no ambiguity since they are the only unknown residue at cycle 1 and 3 respectively. Amino acids Xx1 and Xx4 were assigned from their relative amounts. The presence of a methionine would permit the CNBr cleavage follow by peptide identified by MS/MS.

Probing of the Random Sequence Library

About 130,000 individual beads representing one copy of the library, estimated to be contained in 1.1 ml of settled beads, was hydrated in buffer A (50 mM Na acetate, 0.1% Triton X-100, 0.4 M urea, 0.02% Na azide, 1 mg/ml BSA, pH 3.5; filtered with a 5 micron filter). The beads were soaked in 3% BSA in buffer A for 1 h to block the non-specific binding, and rinsed twice with the same buffer. Recombinant memapsin 2 was diluted into buffer A to 4 nM and incubated with the library for 1 h. A single stringency wash was performed which included 6.7 μM transition-state isosteric inhibitor OM99-2 in buffer B (50 mM Na acetate, 0.1% Triton X-100, 0.02% Na azide, 1 mg/ml BSA, pH 5.5; filtered with 5 micron filter), followed by two additional washes with buffer B containing no OM99-2. Affinity-purified IgG specific for recombinant memapsin 2 was diluted 100 fold in buffer B and incubated 30 min with the library. Following three washes with buffer B, affinity-purified anti-goat-alkaline phosphatase conjugate was diluted into buffer B (1:200) and incubated for 30 min, with three subsequent washes. A single tablet of alkaline phosphatase substrate (BCIP/NBT) was dissolved in 10 ml water and 1 ml applied to the beads and incubated 1 h. Beads were resuspended in 0.02% sodium azide in water and examined under a dissecting microscope. Darkly-stained beads were graded by sight, individually isolated, stripped in 8 M urea for 24 h, and destained in DMF. The sequence determination of the beads were carried out in an Applied Biosystem Protein Sequencer at the Molecular Biology Resource Center on campus and the PTH-amino acids were quantified using reversed-phase HPLC.

Determination of Kinetic Parameters

The kinetic parameters, Kₘ and k_{cat}, using single peptide substrate, and Kᵢ against free inhibitors, were determined as described above.

Model Building of OM00-3 Binding to Memapsin 2

The crystal structure of OM99-2 in complex with memapsin 2 was used as the initial model. Corresponding subsite residues were replaced with that of OM00-3. The side chain conformations were visually selected from a rotomer library for the best fitting into the binding pockets in terms of the non-bonded interaction. Energy minimization was then carried out with the CNS program using Engh and Huber energy parameters (Engh, R. A. and Huber “Accurate bond and angle parameters for X-ray structure refinement” in: Acta Cryst. A47, 392-400 (1991)). C_{Ò} atoms and the atoms with a distance of more than 5 angstroms from the inhibitor were subject to harmonic restrains during the minimization process.

Results

Determination of Substrate Side-Chain Preference in Memapsin 2 Sub-Sites

The substrate cleft of memapsin 2 accommodates eight sub-sites for the side chains as shown in the crystal structure. A complete set of side-chain preference analyzed by classical enzyme kinetics for all sub-sites of memapsin 2 would require the determination of 160 pairs of individual k_{cat} and Kₘ values, a tedious task so far not attained for any aspartic protease with broad specificity. The sub-site preference is, however, defined by the relative catalytic efficiency, k_{cat}/Kₘ values of substrate with different side chains, which may be determined from the relative initial hydrolysis rates of defined mixtures of substrates (Fersht, A. Enzyme Structure and Mechanism, 2^{nd} Ed., Freeman, New York, (1985), the teachings of which are incorporated herein in their entirety). This method has been successfully used to analyze the specificity of other aspartic proteases (Kassel et al., 1995; Koelsch et al., 2000, the teachings of which are incorporated herein in their entirety). The rate determination was further simplified by the use of MALDI-TOF/MS ion intensities for quantification of relative amounts of products and substrates. MALDI-TOF/MS ion intensities have been used to quantify compounds from plasma and cell culture (Sugiyama et al, “A quantitative analysis of serum sulfatide by matrix-assisted laser desorption ionization time-of-flight mass spectrometry with delayed ion extraction” in: Anal. Biochem. 274: 90-97 (1999); Wu et al., “An automated MALDI mass spectrometry approach for optimizing cyclosporin extraction and quantitation” in: Anal Chem. 69: 3767-71 (1997), the teachings of all of which are incorporated herein in their entirety) and pertinently, to determine relative amount of Aβ 40 and Aβ 42 from cell culture (Davies et al., “The structure and function of the aspartic proteinases” in: Annu Rev Biophys Biophys Chem. 19: 189-215 (1990), the teachings of which are incorporated herein in their entirety). Advantages of MALDI-TOF/MS for this method include its sensitivity and rapid acqusition of data. Linearity of ion intensity data with mixtures of product and substrate produced excellent correlation, was consistent for each substrate in the mixture, and required no correction factor. Initial rates of hydrolysis for each peptide in each mixture were subsequently determined using this method. Ratios of these initial rates are proportional to their relative catalytic efficiencies, k_{cat}/Kₘ (Fersht, 1985).

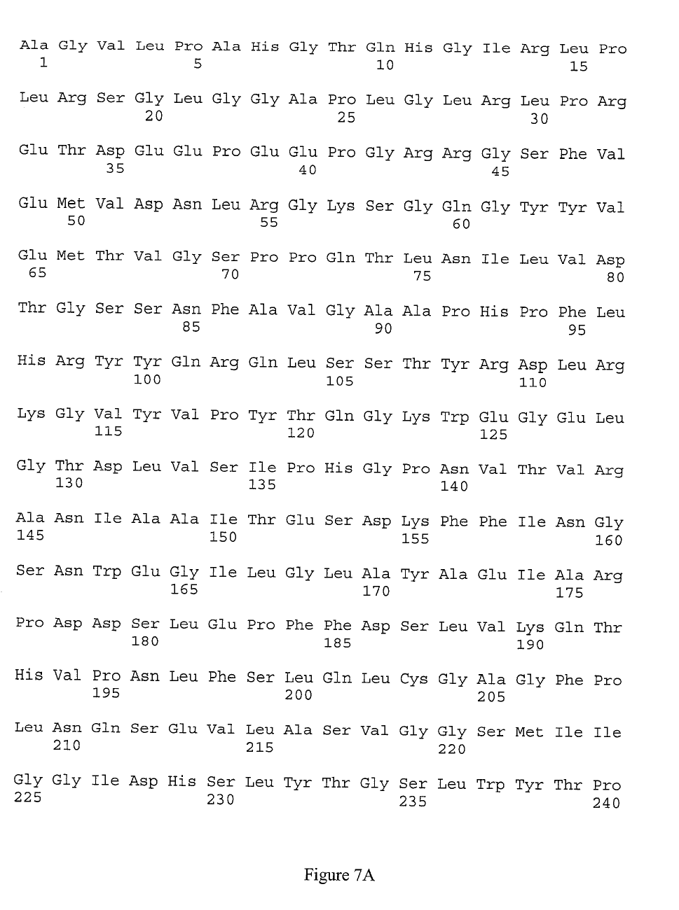

The relative catalytic efficiencies of memapsin 2 for eight sub-site positions of the substrate are presented in FIG. 5. These results confirm previous observations that none of the eight sub-site of memapsin 2 is absolutely stringent. On both the P side and the P′ side, the central sub-sites (P₁ and P₁′) are more stringent than the outside sub-sites (P₄ and P₄′). This is in evidence when the hydrolysis efficiency of the non-preferred residues (background) are compared to the preferred residues. The lack of stringency is more pronounced for the 4 sub-sites on the P′ side, especially for P₃′ and P₄′ where the backgrounds are relatively high. The poor stringencies of these two sub-sites is consistent with the observation that the P₃′ and P₄′ side chains of OM99-2 do not significantly interact with the enzyme in the the crystal structure.

Sub-site P₄ favors Glu over Gln which, in turn, is favored over Asp. P₄-Glu of OM99-2 fits well in S4 pocket with multiple interactions. The reduction of catalytic efficiency from substitution of P₄-Glu with Gln may be due to the loss of charge interaction of P₄ side chain with Arg²³⁵ and Arg³⁰⁷. The further decrease of catalytic efficiency from substitution of P₄-Glu with Asp is likely due to the absence of the hydrogen bond to P₂-Asn. For sub-site P3, an Ile is more preferable than Val as in OM99-2. This is due to a better fitting in the S₃ hydrophobic pocket.

Inhibitor Side Chain Preference Determined from a Combinatorial Library

The sub-site preference for inhibitor binding was also studied. A combinatorial library of approximately 1.3×10⁵ different inhibitors immobilized on beads was synthesized and probed with memapsin 2. The base sequence of the library was taken from OM99-2, EVNL*AAEF (SEQ ID NO: 69) (* designates isostere hydroxyethylene), in which the sub-sites P₃, P₂, P₂′, and P₃′ (boldface) were randomized with all amino acids except cysteine. P₁ and P₁′ were keep constant due to the use of L*A in library synthesis. P4 and P4′ were not randomized in order to keep the library size manageable. Also, the information on those outside sub-sites are less critical for the design of smaller inhibitors. After enhancing memapsin 2 binding selectivity to the library by washing with urea solutions, the bound memapsin 2 on beads was detected by antibody raised to memapsin 2 and an alkaline phophatase conjugated secondary antibody. About 65 from about 130,000 beads were intensely stained as observed under the light microscope. The inhibitor sequences from the 10 most intensely stained beads were determined by automated Edman sequencing (Table 2). A clear consensus at the four randomized positions (boldface) was observed to be ELDLAVEF (SEQ ID NO: 70). The clear consensus in positive beads and the lack of consensus in negative beads (Table 2) supports the contention that the memapsin 2 was bound to preferred sequences in the library. Also, the consensus inhibitor sequence agree well with the results obtained from the substrate studies. An inhibitor, OM00-3, having the consensus sequence ELDL*AVEF (SEQ ID NO: 71) was synthesized using the method described above. The Kᵢ of this inhibitor proved to be 0.31 nM, nearly five fold lower than the Kᵢ of OM99-2.

TABLE 2
Selection of potent competitors of OM99-2 from combinatorial library^{a}

| ID | P3 | P2 | P2′ | P3′ | SEQ ID NO. |
|---|---|---|---|---|---|
| 1 | Leu | Asp | Val | Glu | 72 |
| 2 | Leu | Glu | Val | Glu | 73 |
| 3 | Leu | Asp | Val | Glu | 74 |
| 4 | Leu | Asp | Val | Glu | 75 |
| 5 | Leu | Asp | Val | Gln | 76 |
| 6 | Ile | Asp | Ala | Gln | 77 |
| 7 | Ile | Asp | Val | Tyr | 78 |
| 8 | Leu | Glu | Val | Gln | 79 |
| 9 | Leu | Phe | Val | Glu | 80 |
| 10 | Phe/Ile | Ser | Val | Phe/Ile | 81 |
| Neg1 | Phe | Met | Asn | Arg | 82 |
| Neg2 | Asp | Phe | | Ser | 83 |

^{a}Library template: Gly-P₃-P₂-Leu*Ala-P₂′-P₃′-Phe-Arg-Met-Gly-Gly-Resin (SEQ ID NOS: 72-83 )

Binding of OM00-3 in the Active Site of Memapsin 2

Comparisons between the crystal structure of OM99-2 and the molecular model of OM2000-1 reveal improved inhibitor binding of the later to memapsin 2. As compared to OM99-2, the larger hydrophobic side chains at P₃ and P₂′ subsites for OM00-3 are better accommodated by the enzyme subsite through improved van der Waal interactions. Among these are six significant new interactions: P₃-Lew of inhibitor with Leu and Gly of the enzyme and P₂′-Val with Val, Tyr, Ile and Tyr. In addition, the model shows that replacement of Asn by Asp at the P₂ position introduces three new salt bridges between the side chain atoms CD1 and CD2 of P₂-Asp and that of NE and NH1 of Arg-235. The inter-atomic distances of these salt bridges are 3.6, 3.4 and 3.5 angstroms respectively. These charge interactions should render higher free energies of binding than that of P₂-Asn in OM99-2. These observed structural differences between the binding of two inhibitors are consistent with the 4.5 fold decrease in Kᵢ values from OM99-2 to OM00-3.

To be useful for clinical treatment of AD, memapsin 2 inhibitors must penetrate the blood-brain barrier, and consequently, must be small in size (<700 daltons). An eight-residue inhibitor such as OM00-3, is 917 daltons. Smaller inhibitors can be designed using fewer sub-sites, perhaps spanning only 4 or 5 sub-sites. Information described herein on sub-site preferences together with the information on the three-dimensional structure of the sub-sites can be used in the design of these inhibitors.

### EXAMPLE 6

### Using The Crystal Structure to Design Inhibitors

Pharmaceutically acceptable inhibitor drugs are normally less than 800 daltons. In the case of memapsin 2 inhibitors, this requirement may even be more stringent due to the need for the drugs to penetrate the blood-brain. In the current model, well defined subsite structures at P₄ to P₂′ provide sufficient template areas for rational design of such drugs. The spacial relationships of individual inhibitor side chain with the corresponding subsite of the enzyme as revealed in this crystal structure permits the design of new inhibitor structures in each of these positions. It is also possible to incorporate the unique conformation of subsites P₂′, P₃′ and P₄′ into the selectivity of memapsin 2 inhibitors. The examples of inhibitor design based on the current crystal structure are given below.

### Example A

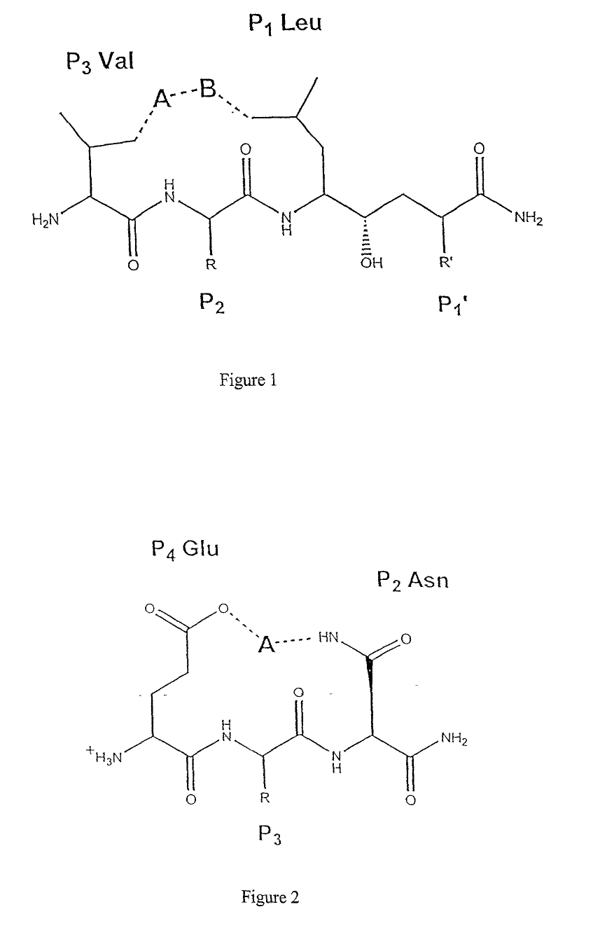

Since the side chains of P₃ Val and P₁ Leu are packed against each other and there is no enzyme structure between them, cross-linking these side chains would increase the binding strength of inhibitor to memapsin 2. This is because when binding to the enzyme, the cross-linked inhibitors would have less entropy difference between the free and bound forms than their non-cross-linked counterparts (Khan, A. R., et al., Biochemistry, 37:16839 (1998), the teachings of which are incorporated herein in their entirety). Possible structures of the cross-linked side chains include those shown in FIG. 1.

### Example B

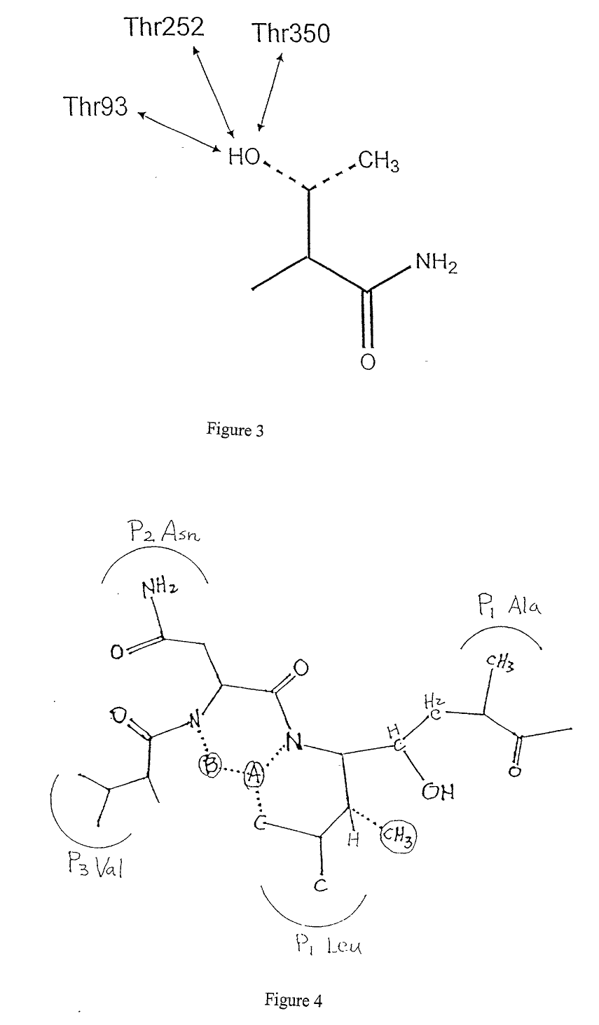

The same situation exits between the P4 Glu and P2 Asn. The current crystal structure shows that these side chains are already hydrogen bonded to each other so the cross linking between them would also derive binding benefit as described in A. The cross-linked structures include those shown in FIG. 2.

### Example C

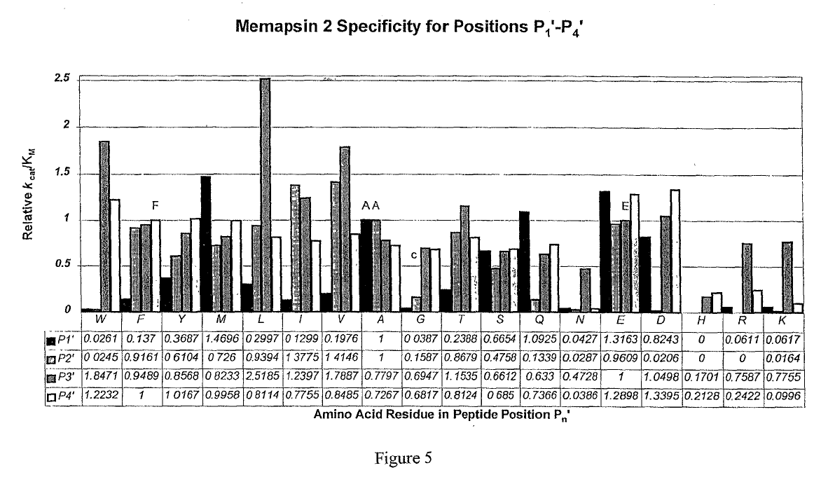

Based on the current crystal structure, the P1′ Ala side chain may be extended to add new hydrophobic, Van der Waals and H-bond interactions. An example of such a design is diagramed in FIG. 3.

### Example D

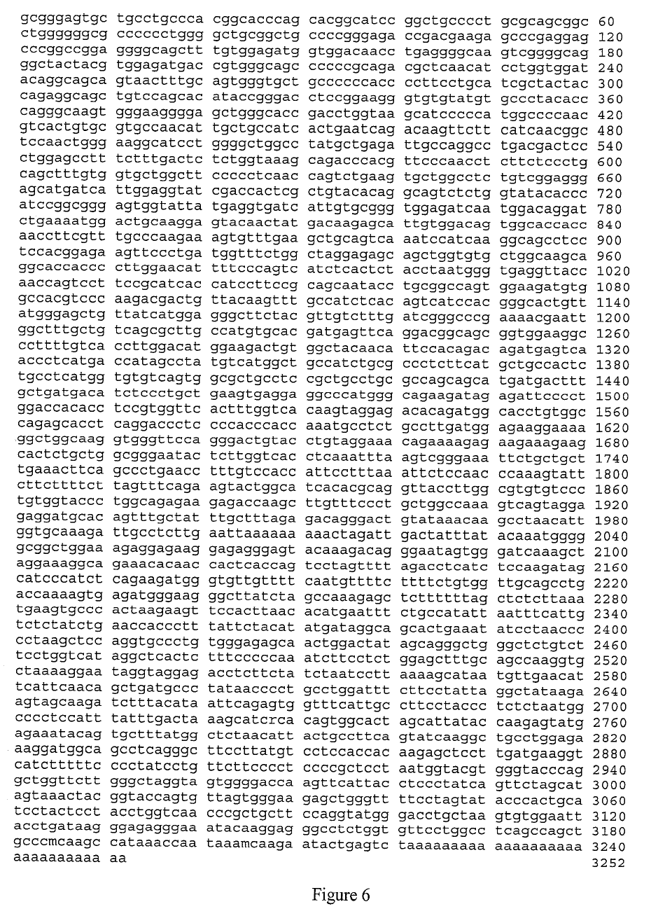

Based on the current crystal structure, the polypeptide backbone in the region of P1, P2, and P3, and the side chain of P1-Leu can be bridged into rings by the addition of two atoms (A and B in FIG. 4). Also, a methyl group can be added to the beta-carbon of the P1-Leu (FIG. 4).

Modifications and variations of the methods and materials described herein will be obvious to those skilled in the art and are intended to come within the scope of the appended claims.

While this invention has been particularly shown and described with references to preferred embodiments thereof, it will be understood by those skilled in the art that various changes in form and details may be made therein without departing from the scope of the invention encompassed by the appended claims.

What is claimed is:

1. An inhibitor of catalytically active memapsin 2 which binds to the active site of the memapsin 2 defined by the presence of two catalytic aspartic residues and substrate binding cleft, the inhibitor having an Kᵢ of less than or equal to 10⁻⁷ M.

2. The inhibitor of claim 1, comprising an isostere of the active site of memapsin 2.

3. The inhibitor of claim 2, comprising a molecule having the general form
X-L₄-P₄-L₃-P₃-L₂-P₂-L₁-P₁-L₀-P₁′-L₁′-P₂′-L₂′-P₃′-L₃′-P₄′-L₄′-Y,
wherein Pₓ represents the substrate specificity position relative to the cleavage site which is represented by an -L₀-, and Lₓ represent the linking regions between each substrate specificity position, Pₓ and wherein L₀ is a non-hydrolyzable bond and P₁′ is —R₁CR₃—, wherein R₁ is a group smaller than CH₂OH (side chain of serine), and at least two other P positions are a hydrophobic group.

4. The inhibitor of claim 1 having a Kᵢ of less than or equal to 10⁻⁶ M.

5. The inhibitor of claim 1 having a Kᵢ of less than or equal to 2 nM.

6. The inhibitor of claim 1 having a Kᵢ of less than or equal to 1 nM.

7. The inhibitor of claim 1 having a root mean square difference of less than or equal to 0.5 Å for the side chain and backbone atoms for amino acids 28-441 of SEQ ID NO: 2.

8. The inhibitor of claim 1 which is permeable to the blood brain barrier.

9. The inhibitor of claim 1 which is less than 900 daltons in molecular weight.

10. The inhibitor of claim 1 which blocks cleavage by memapsin 2 under physiological conditions.

11. The inhibitor of claim 1 having a Kᵢ of less than or equal to 10⁻⁶ M.

12. A compound selected from the group consisting of MMI-005, MMI-012, MMI-017, MMI-018, MMI-025, MMI-026, MMI-037, MMI-039, MMI-040, MMI-065, MMI-066, MMI-070, and MMI-071.

13. A compound selected from the group consisting of MMI-012, MMI-017, MMI-018, MMI-026, MMI-037, MMI-039, MMI-040, MMI-070 and MMI-071.

14. A method for treating a patient to decrease the likelihood of developing or the progression of Alzheimer's disease comprising administering to the individual an effective amount of an inhibitor of memapsin 2 selected from the group consisting of MMI-005, MMI-012, MMI-017, MMI-018, MMI-025, MMI-026, MMI-037, MMI-039, MMI-040, MMI-065, MMI-070 and MMI-071.

15. The method of claim 14, wherein the inhibitor is administered orally.

16. The method of claim 14, wherein the inhibitor blocks cleavage of APP.

17. A method of determining the substrate side-chain preference in memapsin 2 sub-sites, comprising the steps of:
a) reacting a mixture of memapsin 2 substrates with memapsin 2; and
b) determining the sub-site preference of memapsin 2 by determining relative initial hydrolysis rates of the mixture of memapsin 2 substrates.

18. The method of claim 17, wherein the initial hydrolysis rate is determined by using a MALDI-TOF/MS device wherein the ion intensities from the MALDI-TOF/MS measurements are used for quantification of relative amounts of the substrates and the products formed thereof by hydrolysis of the substrates.

19. A method of determining the substrate side-chain preference in memapsin 2 sub-sites, comprising the steps of:
a) preparing a combinatorial library of memapsin 2 inhibitors wherein the inhibitors comprise a base sequence taken from OM99-2;
b) probing the library of inhibitors with memapsin 2 wherein the memapsin 2 may bind one or a plurality of inhibitors to generate one or a plurality of bound memapsin 2; and
c) detecting the bound memapsin 2 with an antibody raised to memapsin 2 and an alkaline phosphatase conjugated secondary antibody.

20. The method of claim 19, wherein the inhibitors are immobilized on beads.

21. The method of claim 19, wherein the base sequence is EVNL*AAEF and wherein the P₃, P₂, P₂′, and P₃′ sub-sites of memapsin 2 are randomized.

22. A method of treating a human suffering from Alzheimer's disease comprising administering to the human an inhibitor of catalytically active memapsin 2 which binds to the active site of the memapsin 2 defined by the presence of two catalytic aspartic residues and substrate binding cleft, the inhibitor having an Kᵢ of less than or equal to 10⁻⁷ M.

23. A method of treating a human suffering from Alzheimer's disease comprising administering to the human an inhibitor of catalytically active memapsin 2 which binds to the active site of the memapsin 2 defined by the presence of two catalytic aspartic residues and substrate binding cleft, the inhibitor having an Kᵢ of less than or equal to 10⁻⁷ M, wherein the inhibitor has a root mean square difference of less than or equal to 0.5 Å for the side chain and backbone atoms for amino acids 28-441 of SEQ ID NO: 2.

24. A method of treating a human suffering from Alzheimer's disease comprising administering to the human a compound selected from the group consisting of MMI-012, MMI-017, MMI-018, MMI-026, MMI-037, MMI-039, MMI-040, MMI-070 and MMI-071.
